(19) United States
(12) Patent Application Publication
(10) Pub. No.: US 2004/0146908 A1
(43) Pub. Date: Jul. 29, 2004

(54) Fibroblast growth factor-19 (FGF-19) nucleic acids and polypeptides and methods of use for the treatment of obesity and related disorders

(75) Inventors: Sean Adams, Randolph Township, NJ (US); Audrey Goddard, San Francisco, CA (US); Austin L. Gurney, Belmont, CA (US); Timothy A. Stewart, San Francisco, CA (US); Elizabeth Tomlinson, Griswold, CT (US); Xing Xian Yu, Carlsbad, CA (US)

Correspondence Address: GENENTECH, INC., 1 DNA WAY, SOUTH SAN FRANCISCO, CA, 94080, US

(73) Assignee: Genentech, Inc.

(21) Appl. No.: 10/712,560

(22) Filed: Nov. 12, 2003

Related U.S. Application Data
(63) Continuation of application No. 09/924,647, filed on Aug. 7, 2001, now abandoned.
Application No. 09/924,647 is a continuation-in-part of application No. 09/767,609, filed on Jan. 22, 2001, now abandoned.
Application No. 09/767,609 is a continuation-in-part of application No. 09/522,342, filed on Mar. 9, 2000, now abandoned.
Application No. 09/522,342 is a continuation-in-part of application No. 09/284,663, filed on Apr. 15, 1999, now abandoned.
Application No. 09/284,663 is a continuation of application No. 09/158,342, filed on Sep. 21, 1998, now abandoned.
Application No. 09/284,663 is a continuation of application No. PCT/US98/25190, filed on Nov. 25, 1998.
(60) Provisional application No. 60/066,840, filed on Nov. 25, 1997.

(30) Foreign Application Priority Data
Sep. 8, 1999 (WO) PCT/US99/20594
Sep. 15, 1999 (WO) PCT/US99/21090
Dec. 20, 1999 (WO) PCT/US99/30999
Feb. 22, 2000 (WO) PCT/US00/04414

(51) Int. Cl. C12Q 1/68; C07H 21/04; C07K 14/50

(52) U.S. Cl. 435/6; 435/69.1; 435/320.1; 435/325; 530/399; 536/23.5

(57) ABSTRACT

The present invention is directed to novel polypeptides belonging to the fibroblast growth factor family and to nucleic acid molecules encoding those polypeptides. Also provided herein are vectors and host cells comprising those nucleic acid sequences, chimeric polypeptide molecules comprising the polypeptides of the present invention fused to heterologous polypeptide sequences, antibodies which bind to the polypeptides of the present invention and to methods for producing the polypeptides of the present invention. Furthermore, methods of treating obesity are provided.

### RELATED APPLICATION

This is a continuation application claiming priority to U.S. application Ser. No. 09/924,647, filed Aug. 7, 2001, which is a continuation-in-part of U.S. application Ser. No. 09/767,609, filed Jan. 22, 2001, now abandoned, which is a continuation-in-part of U.S. application Ser. No. 09/522,342, filed Mar. 9, 2000, which is a continuation-in-part of U.S. application Ser. No. 09/284,663, filed Apr. 15, 1999, now abandoned, which is a continuation of U.S. application Ser. No. 09/158,342, filed Sep. 21, 1998, now abandoned, and to International Application Numbers PCT/US/98/25190, filed Nov. 25, 1998, PCT/US99/20594, filed Sep. 8, 1999, now abandoned, PCT/US99/21090, filed Sep. 15, 1999, now abandoned, PCT/US99/30999, filed Dec. 20, 1999, now abandoned, and PCT/US00/04414, filed Feb. 22, 2000, now abandoned, which applications priority is claimed under 35 USC § 120, and to provisional application Serial No. 60/066,840, filed Nov. 25, 1997, now abandoned, to which application priority is claimed under 35 USC § 119, the entire disclosures of which are hereby incorporated by reference.

### FIELD OF THE INVENTION

The present invention relates generally to the identification and isolation of novel DNA and to the recombinant production of novel polypeptides designated herein as fibroblast growth factor-19 (FGF-19) polypeptides, and to methods, compositions and assays utilizing such polypeptides for the therapeutic treatment of obesity and related disorders and for producing pharmaceutically active materials having therapeutic and pharmacologic properties including those associated with the treatment of obesity and related disorders.

### BACKGROUND OF THE INVENTION

Obesity is a chronic disease that is highly prevalent in modern society and is associated not only with a social stigma, but also with decreased life span and numerous medical problems, including adverse psychological development, reproductive disorders such as polycystic ovarian disease, dermatological disorders such as infections, varicose veins, Acanthosis nigricans, and eczema, exercise intolerance, diabetes mellitus, insulin resistance, hypertension, hypercholesterolemia, cholelithiasis, osteoarthritis, orthopedic injury, thromboembolic disease, cancer, and coronary heart disease. Rissanen et al., British Medical Journal, 301: 835-837 (1990).

Existing therapies for obesity include standard diets and exercise, very low calorie diets, behavioral therapy, pharmacotherapy involving appetite suppressants, thermogenic drugs, food absorption inhibitors, mechanical devices such as jaw wiring, waist cords and balloons, and surgery. Jung and Chong, Clinical Endocrinology, 35: 11-20 (1991); Bray, Am. J. Clin. Nutr., 55: 538S-544S (1992). Protein-sparing modified fasting has been reported to be effective in weight reduction in adolescents. Lee et al., Clin. Pediatr., 31: 234-236 (April 1992). Caloric restriction as a treatment for obesity causes catabolism of body protein stores and produces negative nitrogen balance. Protein-supplemented diets, therefore, have gained popularity as a means of lessening nitrogen loss during caloric restriction. Because such diets produce only modest nitrogen sparing, a more effective way to preserve lean body mass and protein stores is needed. In addition, treatment of obesity would be improved if such a regimen also resulted in accelerated loss of body fat. Various approaches to such treatment include those discussed by Weintraub and Bray, Med. Clinics N. Amer., 73: 237 (1989); Bray, Nutrition Reviews, 49: 33 (1991).

Considering the high prevalence of obesity in our society and the serious consequences associated therewith as discussed above, any therapeutic drug potentially useful in reducing weight of obese persons could have a profound beneficial effect on their health. There is a need in the art for a drug that will reduce total body weight of obese subjects toward their ideal body weight without significant adverse side effects and that will help the obese subject maintain the reduced weight level.

It is therefore desirable to provide a treatment regimen that is useful in returning the body weight of obese subjects toward a normal, ideal body weight.

It is further desirable to provide a therapy for obesity that results in maintenance of the lowered body weight for an extended period of time.

It is also desirable prevent obesity and, once treatment has begun, to arrest progression or prevent the onset of diseases that are the consequence of, or secondary to, the obesity, such as arteriosclerosis and polycystic ovarian disease.

Such methods of treatment and related compositions are provided herein. Also provided herein are novel proteins and nucleic acids, and methods for screening for modulators of the same. Other methods, treatments and compositions provided herein will become apparent to the skilled artisan.

### SUMMARY OF THE INVENTION

A cDNA clone (designated herein as DNA49435-1219) has been identified that encodes a novel polypeptide, which has some sequence similarity to members of the fibroblast growth factor family, designated in the present application as “fibroblast growth factor-19” (FGF-19).

In one embodiment, the invention provides an isolated nucleic acid molecule comprising a nucleotide sequence that encodes a FGF-19 polypeptide.

In one aspect, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule encoding a polypeptide having the sequence of amino acid residues from about 1 or about 23 to about 216, inclusive, of FIG. 2 (SEQ ID NO:2), or (b) the complement of the DNA molecule of (a).

In another aspect, the isolated nucleic acid molecule comprises (a) a nucleotide sequence encoding a FGF-19 polypeptide having the sequence of amino acid residues from about 1 or about 23 to about 216, inclusive, of FIG. 2 (SEQ ID NO:2), or (b) the complement of the nucleotide sequence of (a).

In other aspects, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule having the sequence of nucleotides from about 464 or about 530 to about 1111, inclusive, of FIG. 1 (SEQ ID NO:1), or (b) the complement of the DNA molecule of (a).

In another aspect, the isolated nucleic acid molecule comprises (a) the nucleotide sequence of from about 464 or about 530 to about 1111, inclusive, of FIG. 1 (SEQ ID NO:1), or (b) the complement of the nucleotide sequence of (a).

In a further aspect, the invention concerns an isolated nucleic acid molecule comprising a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule that encodes the same mature polypeptide encoded by the human protein cDNA deposited with the ATCC on Nov. 21, 1997 under ATCC Deposit No. 209480 (DNA49435-1219) or (b) the complement of the DNA molecule of (a). In a preferred embodiment, the isolated nucleic acid molecule comprises (a) a nucleotide sequence encoding the same mature polypeptide encoded by the human protein cDNA deposited with the ATCC on Nov. 21, 1997 under ATCC Deposit No. 209480 (DNA49435-1219) or (b) the complement of the nucleotide sequence of (a).

In another aspect, the invention concerns an isolated nucleic acid molecule comprising a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) the full-length polypeptide coding sequence of the human protein cDNA deposited with the ATCC on Nov. 21, 1997 under ATCC Deposit No. 209480 (DNA49435-1219) or (b) the complement of the nucleotide sequence of (a). In a preferred embodiment, the isolated nucleic acid molecule comprises (a) the full-length polypeptide coding sequence of the DNA deposited with the ATCC on Nov. 21, 1997 under ATCC Deposit No. 209480 (DNA49435-1219) or (b) the complement of the nucleotide sequence of (a).

In another aspect, the invention concerns an isolated nucleic acid molecule which encodes an active FGF-19 polypeptide as defined below comprising a nucleotide sequence that hybridizes to the complement of a nucleic acid sequence that encodes amino acids 1 or about 23 to about 216, inclusive, of FIG. 2 (SEQ ID NO:2). Preferably, hybridization occurs under stringent hybridization and wash conditions.

In yet another aspect, the invention concerns an isolated nucleic acid molecule which encodes an active FGF-19 polypeptide as defined below comprising a nucleotide sequence that hybridizes to the complement of the nucleic acid sequence between about nucleotides 464 or about 530 and about 1111, inclusive, of FIG. 1 (SEQ ID NO:1). Preferably, hybridization occurs under stringent hybridization and wash conditions.

In a further aspect, the invention concerns an isolated nucleic acid molecule having at least about 22 nucleotides and which is produced by hybridizing a test DNA molecule under stringent conditions with (a) a DNA molecule encoding a FGF-19 polypeptide having the sequence of amino acid residues from about 1 or about 23 to about 216, inclusive, of FIG. 2 (SEQ ID NO:2), or (b) the complement of the DNA molecule of (a), and, if the test DNA molecule has at least about an 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) or (b), and isolating the test DNA molecule.

In another aspect, the invention concerns an isolated nucleic acid molecule comprising (a) a nucleotide sequence encoding a polypeptide scoring at least about 80% positives, alternatively at least about 81% positives, alternatively at least about 82% positives, alternatively at least about 83% positives, alternatively at least about 84% positives, alternatively at least about 85% positives, alternatively at least about 86% positives, alternatively at least about 87% positives, alternatively at least about 88% positives, alternatively at least about 89% positives, alternatively at least about 90% positives, alternatively at least about 91% positives, alternatively at least about 92% positives, alternatively at least about 93% positives, alternatively at least about 94% positives, alternatively at least about 95% positives, alternatively at least about 96% positives, alternatively at least about 97% positives, alternatively at least about 98% positives and alternatively at least about 99% positives when compared with the amino acid sequence of residues about 1 or about 23 to 216, inclusive, of FIG. 2 (SEQ ID NO:2), or (b) the complement of the nucleotide sequence of (a).

In a specific aspect, the invention provides an isolated nucleic acid molecule comprising DNA encoding a FGF-19 polypeptide without the N-terminal signal sequence and/or the initiating methionine, or is complementary to such encoding nucleic acid molecule. The signal peptide has been tentatively identified as extending from about amino acid position 1 to about amino acid position 22, inclusive, in the sequence of FIG. 2 (SEQ ID NO:2). It is noted, however, that the C-terminal boundary of the signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10: 1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These polypeptides, and the polynucleotides encoding them, are contemplated by the present invention. As such, for purposes of the present application, the signal peptide of the FGF-19 polypeptide shown in FIG. 2 (SEQ ID NO:2) extends from amino acids 1 to X of FIG. 2 (SEQ ID NO:2), wherein X is any amino acid from 17 to 27 of FIG. 2 (SEQ ID NO:2). Therefore, mature forms of the FGF-19 polypeptide which are encompassed by the present invention include those comprising amino acids X to 216 of FIG. 2 (SEQ ID NO:2), wherein X is any amino acid from 17 to 27 of FIG. 2 (SEQ ID NO:2) and variants thereof as described below. Isolated nucleic acid molecules encoding these polypeptides are also contemplated.

Another embodiment is directed to fragments of a FGF-19 polypeptide sequence which includes the coding sequence that may find use as, for example, hybridization probes or for encoding fragments of a FGF-19 polypeptide that may optionally encode a polypeptide comprising a binding site for an anti-FGF-19 antibody. Such nucleic acid fragments are usually at least about 20 nucleotides in length, alternatively at least about 30 nucleotides in length, alternatively at least about 40 nucleotides in length, alternatively at least about 50 nucleotides in length, alternatively at least about 60 nucleotides in length, alternatively at least about 70 nucleotides in length, alternatively at least about 80 nucleotides in length, alternatively at least about 90 nucleotides in length, alternatively at least about 100 nucleotides in length, alternatively at least about 110 nucleotides in length, alternatively at least about 120 nucleotides in length, alternatively at least about 130 nucleotides in length, alternatively at least about 140 nucleotides in length, alternatively at least about 150 nucleotides in length, alternatively at least about 160 nucleotides in length, alternatively at least about 170 nucleotides in length, alternatively at least about 180 nucleotides in length, alternatively at least about 190 nucleotides in length, alternatively at least about 200 nucleotides in length, alternatively at least about 250 nucleotides in length, alternatively at least about 300 nucleotides in length, alternatively at least about 350 nucleotides in length, alternatively at least about 400 nucleotides in length, alternatively at least about 450 nucleotides in length, alternatively at least about 500 nucleotides in length, alternatively at least about 600 nucleotides in length, alternatively at least about 700 nucleotides in length, alternatively at least about 800 nucleotides in length, alternatively at least about 900 nucleotides in length and alternatively at least about 1000 nucleotides in length, wherein in this context the term “about” means the referenced nucleotide sequence length plus or minus 10% of that referenced length. In a preferred embodiment, the nucleotide sequence fragment is derived from any coding region of the nucleotide sequence shown in FIG. 1 (SEQ ID NO:1). It is noted that novel fragments of a FGF-19 polypeptide-encoding nucleotide sequence may be determined in a routine manner by aligning the FGF-19 polypeptide-encoding nucleotide sequence with other known nucleotide sequences using any of a number of well known sequence alignment programs and determining which FGF-19 polypeptide-encoding nucleotide sequence fragment(s) are novel. All of such FGF-19 polypeptide-encoding nucleotide sequences are contemplated herein and can be determined without undue experimentation. Also contemplated are the FGF-19 polypeptide fragments encoded by these nucleotide molecule fragments, preferably those FGF-19 polypeptide fragments that comprise a binding site for an anti-FGF-19 antibody.

In another embodiment, the invention provides a vector comprising a nucleotide sequence encoding FGF-19 or its variants. The vector may comprise any of the isolated nucleic acid molecules hereinabove identified.

A host cell comprising such a vector is also provided. By way of example, the host cells may be CHO cells, E. coli, baculovirus infected insect cells, or yeast. A process for producing FGF-19 polypeptides is further provided and comprises culturing host cells under conditions suitable for expression of FGF-19 and recovering FGF-19 from the cell culture.

In another embodiment, the invention provides isolated FGF-19 polypeptide encoded by any of the isolated nucleic acid sequences hereinabove identified.

In a specific aspect, the invention provides isolated native sequence FGF-19 polypeptide, which in certain embodiments, includes an amino acid sequence comprising residues from about 1 or about 23 to about 216 of FIG. 2 (SEQ ID NO:2).

In another aspect, the invention concerns an isolated FGF-19 polypeptide, comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to the sequence of amino acid residues from about 1 or about 23 to about 216, inclusive, of FIG. 2 (SEQ ID NO:2).

In a further aspect, the invention concerns an isolated FGF-19 polypeptide comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to an amino acid sequence encoded by the human protein cDNA deposited with the ATCC on Nov. 21, 1997 under ATCC Deposit No. 209480 (DNA49435-1219). In a preferred embodiment, the isolated FGF-19 polypeptide comprises an amino acid sequence encoded by the human protein cDNA deposited with the ATCC on Nov. 21, 1997 under ATCC Deposit No. 209480 (DNA49435-1219).

In a further aspect, the invention concerns an isolated FGF-19 polypeptide comprising an amino acid sequence scoring at least about 80% positives, alternatively at least about 81% positives, alternatively at least about 82% positives, alternatively at least about 83% positives, alternatively at least about 84% positives, alternatively at least about 85% positives, alternatively at least about 86% positives, alternatively at least about 87% positives, alternatively at least about 88% positives, alternatively at least about 89% positives, alternatively at least about 90% positives, alternatively at least about 91% positives, alternatively at least about 92% positives, alternatively at least about 93% positives, alternatively at least about 94% positives, alternatively at least about 95% positives, alternatively at least about 96% positives, alternatively at least about 97% positives, alternatively at least about 98% positives and alternatively at least about 99% positives when compared with the amino acid sequence of residues from about 1 or about 23 to about 216, inclusive, of FIG. 2 (SEQ ID NO:2).

In a specific aspect, the invention provides an isolated FGF-19 polypeptide without the N-terminal signal sequence and/or the initiating methionine and is encoded by a nucleotide sequence that encodes such an amino acid sequence as hereinbefore described. Processes for producing the same are also herein described, wherein those processes comprise culturing a host cell comprising a vector which comprises the appropriate encoding nucleic acid molecule under conditions suitable for expression of the FGF-19 polypeptide and recovering the FGF-19 polypeptide from the cell culture.

In yet another aspect, the invention concerns an isolated FGF-19 polypeptide, comprising the sequence of amino acid residues from about 1 or about 23 to about 216, inclusive, of FIG. 2 (SEQ ID NO:2), or a fragment thereof which is biologically active or sufficient to provide a binding site for an anti-FGF-19 antibody, wherein the identification of FGF-19 polypeptide fragments that possess biological activity or provide a binding site for an anti-FGF-19 antibody may be accomplished in a routine manner using techniques which are well known in the art. Preferably, the FGF-19 fragment retains a qualitative biological activity of a native FGF-19 polypeptide, including the ability to therapeutically treat obesity.

In a still further aspect, the invention provides a polypeptide produced by (i) hybridizing a test DNA molecule under stringent conditions with (a) a DNA molecule encoding a FGF-19 polypeptide having the sequence of amino acid residues from about 1 or about 23 to about 216, inclusive, of FIG. 2 (SEQ ID NO:2), or (b) the complement of the DNA molecule of (a), and if the test DNA molecule has at least about an 80% sequence identity, preferably at least about an 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) or (b), (ii) culturing a host cell comprising the test DNA molecule under conditions suitable for expression of the polypeptide, and (iii) recovering the polypeptide from the cell culture.

In another embodiment, the invention provides chimeric molecules comprising a FGF-19 polypeptide fused to a heterologous polypeptide or amino acid sequence, wherein the FGF-19 polypeptide may comprise any FGF-19 polypeptide, variant or fragment thereof as hereinbefore described. An example of such a chimeric molecule comprises a FGF-19 polypeptide fused to an epitope tag sequence or a Fc region of an immunoglobulin.

In another embodiment, the invention provides an antibody as defined below which specifically binds to a FGF-19 polypeptide as hereinbefore described. Optionally, the antibody is a monoclonal antibody, an antibody fragment or a single chain antibody.

In yet another embodiment, the invention concerns agonists and antagonists of a native FGF-19 polypeptide as defined below. In a particular embodiment, the agonist or antagonist is an anti-FGF-19 antibody or a small molecule.

In a further embodiment, the invention concerns a method of identifying agonists or antagonists to a FGF-19 polypeptide which comprise contacting the FGF-19 polypeptide with a candidate molecule and monitoring a biological activity mediated by said FGF-19 polypeptide. Preferably, the FGF-19 polypeptide is a native FGF-19 polypeptide.

In a still further embodiment, the invention concerns a composition of matter comprising a FGF-19 polypeptide, or an agonist or antagonist of a FGF-19 polypeptide as herein described, or an anti-FGF-19 antibody, in combination with a carrier. Optionally, the carrier is a pharmaceutically acceptable carrier.

Another embodiment of the present invention is directed to the use of a FGF-19 polypeptide, or an agonist or antagonist thereof as herein described, or an anti-FGF-19 antibody, for the preparation of a medicament useful in the treatment of a condition which is responsive to the FGF-19 polypeptide, an agonist or antagonist thereof or an anti-FGF-19 antibody.

In one embodiment, a method for screening for a bioactive agent capable of binding to FGF-19 is provided. In one aspect, the method comprises adding a candidate bioactive agent to a sample of FGF-19 and determining the binding of said candidate agent to said FGF-19, wherein binding indicates a bioactive agent capable of binding to FGF-19.

Additionally provided herein is a method for screening for a bioactive agent capable of modulating the activity of FGF-19. In one embodiment, a method is provided which comprises the steps of adding a candidate bioactive agent to a sample of FGF-19 and determining an alteration in the biological activity of FGF-19, wherein an alteration indicates a bioactive agent capable of modulating the activity of FGF-19. In one embodiment, FGF-19 activity is decreased uptake of glucose in cells. In another embodiment, FGF-19 activity is increased leptin release from cells. In a preferred embodiment, FGF-19 activity is decreased uptake of glucose and increased leptin release from cells. Preferably the cells are adipocytes. In yet another embodiment, FGF-19 activity is increased oxidation of lipids and carbohydrates. Preferably the cells are liver or muscle cells.

In yet another embodiment, the invention provides a method of identifying a receptor for FGF-19. In a preferred embodiment, the method comprises combining FGF-19 with a composition comprising cell membrane material wherein said FGF-19 complexes with a receptor on said cell membrane material, and identifying said receptor as a FGF-19 receptor. In one embodiment, the method includes a step of crosslinking said FGF-19 and receptor. The cell membrane can be from an intact cell or a cell membrane extract preparation.

In a further aspect of the invention, a method is provided for inducing leptin release from cells, preferably adipocytes. In one embodiment, the method comprises administering FGF-19 to cells in an amount effective to induce leptin release.

In the methods provided herein, FGF-19 may be administered as a nucleic acid which expresses FGF-19 or in protein form. As further described below, FGF-19 may be administered by infusion or in a sustained release formulation. Preferably, FGF-19 is administered to an individual with a pharmaceutically acceptable carrier.

Also provided herein is a method for inducing a decrease in glucose uptake in cells, preferably adipocyte cells.

In one embodiment the method comprises administering FGF-19 to cells in an amount effective to induce a decrease in glucose uptake.

In yet another aspect of the invention a method of treating an individual for obesity is provided. In one embodiment the method comprises administering to an individual a composition comprising FGF-19 in an amount effective to treat obesity. In this manner, conditions related to obesity can also be treated such as cardiovascular disease.

Also provided herein is a method of reducing total body mass in an individual comprising administering to said individual an effective amount of FGF-19. In a preferred embodiment, adiposity (fat) of an individual is reduced.

Moreover, a method is provided herein for reducing the level of at least one of triglycerides and free fatty acids in an individual comprising administering to said individual an effective amount of FGF-19. Also provided herein is a method of increasing the metabolic rate in an individual comprising administering to said individual an effective amount of FGF-19.

Also provided herein is an animal model for determining the affects of FGF-19 and modulators thereof under varying conditions and states. In one embodiment, an animal, preferably a rodent, is provided which comprises a genome comprising a transgene encoding FGF-19.

### BRIEF DESCRIPTION OF THE DRAWINGS

FIG. 1 shows the nucleotide sequence (SEQ ID NO:1) of a cDNA containing a nucleotide sequence (nucleotides 464-1111) encoding native sequence FGF-19, wherein the nucleotide sequence (SEQ ID NO:1) is a clone designated herein as “DNA49435-1219”. Also presented in bold font and underlined are the positions of the respective start and stop codons.

FIG. 2 shows the amino acid sequence (SEQ ID NO:2) of a native sequence FGF-19 polypeptide as derived from the coding sequence of SEQ ID NO:1. Also shown are the approximate locations of various other important polypeptide domains.

FIGS. 3A and 3B show bar graphs demonstrating that MLC-FGF-19 transgenic mice weigh less than their non-transgenic littermates (FIG. 3A) and have lower circulating leptin levels (FIG. 3B). FIG. 3A shows the weight of FGF-19 transgenic mice (solid bars) and non transgenic (wild-type) littermates (stippled bar) at 6 weeks of age during ad libitum feeding (far left), after 6 and 24 hour fasts, and 24 hours after ending a 24 hour fast (far right). FIG. 3B shows the sera of the same groups of mice represented in FIG. 3A in an assay for leptin (vertical bar).

FIGS. 4A-4D are bar graphs demonstrating that FGF-19 transgenic mice have increased food intake and urine production but have a normal hematocrit. A group of mice were monitored for food intake during ad libitum feeding and 24 hours after ending a 24 hour fast (FIG. 4A), water intake (FIG. 4B), urine output (FIG. 4C) and hematocrit (FIG. 4D) wherein the results for the FGF-19 transgenic mice in each graph are shown by the solid black bar and the results for the wild-type are shown by the stippled bar.

FIG. 5 is a bar graph demonstrating that FGF-19 transgenic mice have an increased rate of oxygen consumption. Oxygen consumption is shown for FGF-19 transgenic mice (solid black bars) and wild-type (stippled bars) during both light cycles (dark or light), following a 24 hour fast and 24 hours after ending a 24 hour fast.

FIGS. 6A and 6B are bar graphs demonstrating that FGF-19 transgenic mice (solid black bars) have decreased triglycerides (FIG. 6A) and free fatty acids (FIG. 6B) over wild-type mice (stippled bars).

FIGS. 7A and 7B are bar graphs which demonstrate that infusing non-transgenic mice with FGF-19 (solid black bars) leads to an increase in food intake (FIG. 7A) and an increase in oxygen consumption (FIG. 7B) over mice infused with vehicle lacking FGF-19 (stippled bars), wherein “n” means night and “d” means day.

FIGS. 8A and 8B are bar graphs indicating that FGF-19 increases leptin release from adipocytes (FIG. 8A) and decreases glucose uptake by adipocytes (FIG. 8B).

FIG. 9 is a bar graph showing the fat pad weight of FGF-19 transgenic mice (shaded bars) or wild-type (solid black bars) each on a high fat diet (HFD) over time, wherein along the horizontal bar starting at the left, the results are shown at 6 weeks for epididymal (HFD Ep) and then for retroperitoneal with peri-renal (HFD RP/PR), and then at 10 weeks for epididymal and then for retroperitoneal with peri-renal.

FIG. 10 is a bar graph showing the glucose tolerance of FGF-19 transgenic mice (shaded bars) or wild-type (solid black bars) over time (both on high fat diets for ten weeks).

FIGS. 11A and 11B are bar graphs showing that recombinant FGF-19 increases metabolic rate. Recombinant human FGF-19 was injected (30 μg/mouse; i.v., twice/day) into chow fed mice. A) The percentage difference in metabolic rate (mean+/−SEM, comparing the FGF-19 treated mice to the control treated mice) is shown for each day and separated into the light and dark cycles. In B) the average metabolic rate over days 4, 5 and 6 is shown (mean+/−SD). For A and B, N=7 (FGF-19) and 8 (control). *, P<0.05.

FIGS. 12A-12D are graphs showing that MLC-FGFG19 transgenic mice eat more, weigh less and have a higher metabolic rate. A. Food intake by transgenic mice on low (LF) or high (HF) fat diets. B. Metabolizable energy for females on the high fat diet. C. Weight gain over the six weeks that the mice were on either the low or high fat diets. Symbols indicating statistical significance have been omitted for clarity. All Tg vs. Wt comparisons (same sex, diet and age) are significant, P<0.01. D. The metabolic rate of the transgenic mice was measured by indirect calorimetry. For A-D, n=4/group. *, P<0.05; #, P<0.01.

FIGS. 13A-13C are bar graphs showing MLC-FGFG19 transgenic mice are resistant to the adipogenic effects of a high fat diet. A. Epididymal (male), uterine (female) and peri-renal (male and female) fat pad weights are shown. B. Leptin levels were determined at the indicated times subsequent to the initiation of either the LF or the HF diet. Data shown are for females. C. Fat content of liver and muscle (soleus) of females is shown. For A-C, n=4/group. *, P<0.05; #, P<0.01.

FIGS. 14A-14D are graphs showing MLC-FGFG19 transgenic mice have improved glucose tolerance and insulin sensitivity. The mice described in FIG. 13 were analyzed after six weeks on the defined diets. A. Glucose levels following a GTT for male mice on the high fat diet (N=4/group). B. Serum insulin levels in ad lib. fed and fasted male mice (N=4/group). C. Glucose levels in male mice on chow (n=6-7, 9 weeks of age) following an insulin injection. D. Radioactivity ratios in male mice as a function of time after injection with ³H 2-deoxyglucose and ¹⁴C sucrose (n=5, age 9 weeks). *, P<0.05; #, P<0.01.

FIGS. 15A-15B are bar graphs showing the metabolic effects of FGF-19 are also present in MT-FGF-19 transgenic mice. Food intake (A) and oxygen consumption (B) in female metallothionein-FGF-19 transgenic mice (n=3 transgenic, 5 wild type). *, P<0.05; #, P<0.01.

FIG. 16 is a bar graph showing the expression levels of α-3 adrenergic receptor in brown adipose tissue (BAT), white adipose tissue (WAT) and liver in FGF-19 transgenic mice. P<0.05 vs. wild type controls.

FIG. 17 is a bar graph showing the metabolic rate of FVB mice (Charles River Labs) during light and dark cycles given an intracerebralventricular (ICV) dose of vehicle or rhFGF-19, once daily for 5 days. P<0.05 vs. vehicle controls with the same cycle on the same day.

FIG. 18 is a bar graph showing the expression levels of Cyp7a in liver in FGF-19 transgenic mice, wild-type mice, FVB mice given an intracerebralventricular (ICV) dose of vehicle or rhFGF-19. P<0.01.

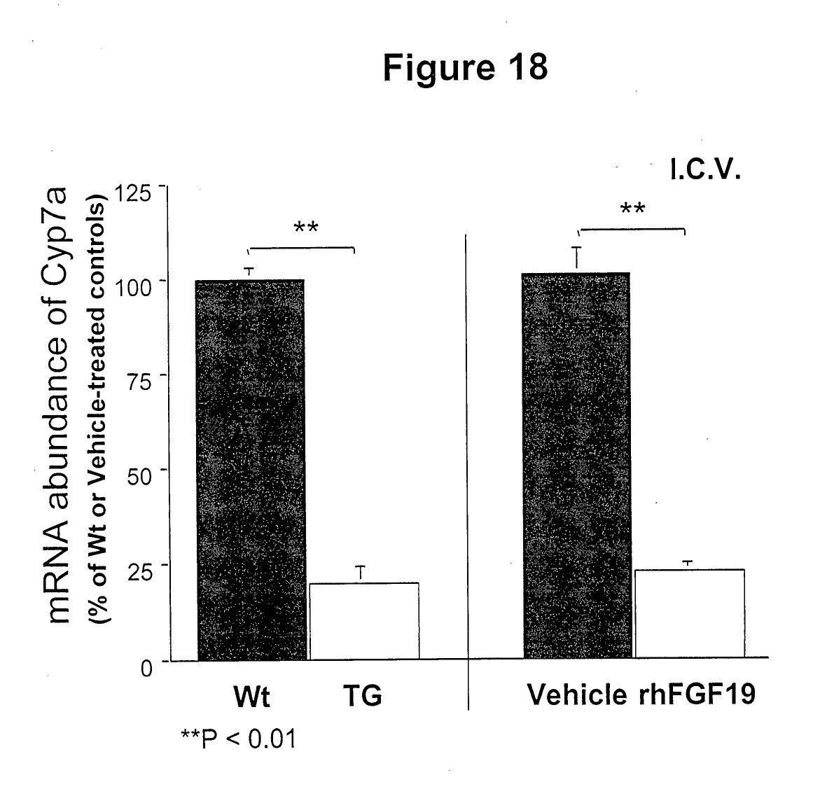

FIG. 19 is a graph showing the metabolic rate of FVB mice (Charles River Labs) during light and dark cycles given an intracerebralventricular (ICV) injection of vehicle or various doses of rhFGF-19, once daily for 5 days.

FIG. 20 is a chart showing the expression levels of neuropeptide Y (NPY), agouti-related protein (AgRP), pro-opiomelanocortin (POMC) and melanocortin 4-receptor (MC4-R) in the hypothalamus of FVB mice (Charles River Labs) given an intracerebralventricular (ICV) injection of vehicle or various doses of rhFGF-19. Numbers in the figure indicate fold-difference in expression relative to vehicle-treated controls.

### DETAILED DESCRIPTION OF THE PREFERRED EMBODIMENTS

I. Definitions

The terms “FGF-19 polypeptide”, “FGF-19 protein” and “FGF-19” when used herein encompass native sequence FGF-19 and FGF-19 polypeptide variants (which are further defined herein). The FGF-19 polypeptide may be isolated from a variety of sources, such as from human tissue types or from another source, or prepared by recombinant and/or synthetic methods.

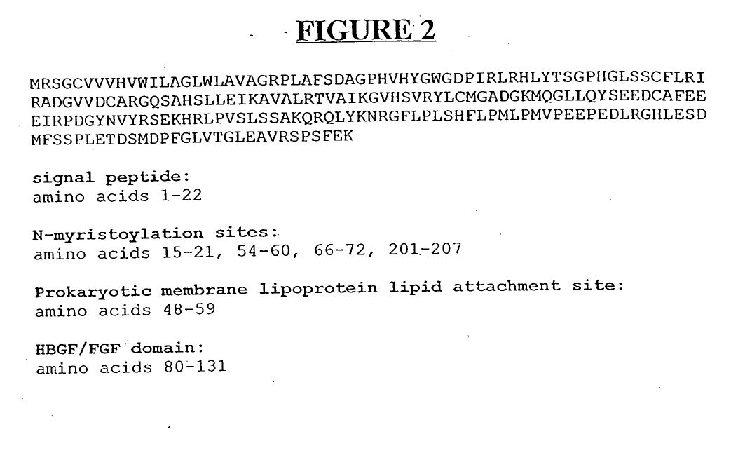

A “native sequence FGF-19” comprises a polypeptide having the same amino acid sequence as a FGF-19 derived from nature. Such native sequence FGF-19 can be isolated from nature or can be produced by recombinant and/or synthetic means. The term “native sequence FGF-19” specifically encompasses naturally-occurring truncated or secreted forms (e.g., an extracellular domain sequence), naturally-occurring variant forms (e.g., alternatively spliced forms) and naturally-occurring allelic variants of the FGF-19. In one embodiment of the invention, the native sequence FGF-19 is a mature or full-length native sequence FGF-19 comprising amino acids 1 to 216 of FIG. 2 (SEQ ID NO:2). Also, while the FGF-19 polypeptide disclosed in FIG. 2 (SEQ ID NO:2) is shown to begin with the methionine residue designated herein as amino acid position 1, it is conceivable and possible that another methionine residue located either upstream or downstream from amino acid position 1 in FIG. 2 (SEQ ID NO:2) may be employed as the starting amino acid residue for the FGF-19 polypeptide.

“FGF-19 variant polypeptide” means an active FGF-19 polypeptide as defined below having at least about 80% amino acid sequence identity with the amino acid sequence of (a) residues 1 or about 23 to 216 of the FGF-19 polypeptide shown in FIG. 2 (SEQ ID NO:2), (b) X to 216 of the FGF-19 polypeptide shown in FIG. 2 (SEQ ID NO:2), wherein X is any amino acid residue from 17 to 27 of FIG. 2 (SEQ ID NO:2), or (c) another specifically derived fragment of the amino acid sequence shown in FIG. 2 (SEQ ID NO:2). Such FGF-19 variant polypeptides include, for instance, FGF-19 polypeptides wherein one or more amino acid residues are added, or deleted, at the—and/or C-terminus, as well as within one or more internal domains, of the sequence of FIG. 2 (SEQ ID NO:2). Ordinarily, a FGF-19 variant polypeptide will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity with (a) residues 1 or about 23 to 216 of the FGF-19 polypeptide shown in FIG. 2 (SEQ ID NO:2), (b) X to 216 of the FGF-19 polypeptide shown in FIG. 2 (SEQ ID NO:2), wherein X is any amino acid residue from 17 to 27 of FIG. 2 (SEQ ID NO:2), or (c) another specifically derived fragment of the amino acid sequence shown in FIG. 2 (SEQ ID NO:2). FGF-19 variant polypeptides do not encompass the native FGF-19 polypeptide sequence. Ordinarily, FGF-19 variant polypeptides are at least about 10 amino acids in length, alternatively at least about 20 amino acids in length, alternatively at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, alternatively at least about 200 amino acids in length, alternatively at least about 300 amino acids in length, or more.

“Percent (%) amino acid sequence identity” with respect to the FGF-19 polypeptide sequences identified herein is defined as the percentage of amino acid residues in a candidate sequence that are identical with the amino acid residues in a FGF-19 sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations, Tables 2 and 3 demonstrate how to calculate the % amino acid sequence identity of the amino acid sequence designated “Comparison Protein” to the amino acid sequence designated “PRO”.

Unless specifically stated otherwise, all % amino acid sequence identity values used herein are obtained as described above using the ALIGN-2 sequence comparison computer program. However, % amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“FGF-19 variant polynucleotide” or “FGF-19 variant nucleic acid sequence” means a nucleic acid molecule which encodes an active FGF-19 polypeptide as defined below and which has at least about 80% nucleic acid sequence identity with either (a) a nucleic acid sequence which encodes residues 1 or about 23 to 216 of the FGF-19 polypeptide shown in FIG. 2 (SEQ ID NO:2), (b) a nucleic acid sequence which encodes amino acids X to 216 of the FGF-19 polypeptide shown in FIG. 2 (SEQ ID NO:2), wherein X is any amino acid residue from 17 to 27 of FIG. 2 (SEQ ID NO:2), or (c) a nucleic acid sequence which encodes another specifically derived fragment of the amino acid sequence shown in FIG. 2 (SEQ ID NO:2). Ordinarily, a FGF-19 variant polynucleotide will have at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity with either (a) a nucleic acid sequence which encodes residues 1 or about 23 to 216 of the FGF-19 polypeptide shown in FIG. 2 (SEQ ID NO:2), (b) a nucleic acid sequence which encodes amino acids X to 216 of the FGF-19 polypeptide shown in FIG. 2 (SEQ ID NO:2), wherein X is any amino acid residue from 17 to 27 of FIG. 2 (SEQ ID NO:2), or (c) a nucleic acid sequence which encodes another specifically derived fragment of the amino acid sequence shown in FIG. 2 (SEQ ID NO:2). FGF-19 polynucleotide variants do not encompass the native FGF-19 nucleotide sequence.

Ordinarily, FGF-19 variant polynucleotides are at least about 30 nucleotides in length, alternatively at least about 60 nucleotides in length, alternatively at least about 90 nucleotides in length, alternatively at least about 120 nucleotides in length, alternatively at least about 150 nucleotides in length, alternatively at least about 180 nucleotides in length, alternatively at least about 210 nucleotides in length, alternatively at least about 240 nucleotides in length, alternatively at least about 270 nucleotides in length, alternatively at least about 300 nucleotides in length, alternatively at least about 450 nucleotides in length, alternatively at least about 600 nucleotides in length, alternatively at least about 900 nucleotides in length, or more.

“Percent (%) nucleic acid sequence identity” with respect to the FGF-19 polypeptide-encoding nucleic acid sequences identified herein is defined as the percentage of nucleotides in a candidate sequence that are identical with the nucleotides in a FGF-19 polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % nucleic acid sequence identity values are obtained as described below by using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or maybe compiled from the source code provided in Table 1. The ALIGN-2 program should be compiled for use on a UNIX operating system, preferably digital UNIX V4.0D. All sequence comparison parameters are set by the ALIGN-2 program and do not vary.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5 demonstrate how to calculate the % nucleic acid sequence identity of the nucleic acid sequence designated “Comparison DNA” to the nucleic acid sequence designated “PRO-DNA”.

Unless specifically stated otherwise, all % nucleic acid sequence identity values used herein are obtained as described above using the ALIGN-2 sequence comparison computer program. However, % nucleic acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.govor otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, FGF-19 variant polynucleotides are nucleic acid molecules that encode an active FGF-19 polypeptide and which are capable of hybridizing, preferably under stringent hybridization and wash conditions, to nucleotide sequences encoding the full-length FGF-19 polypeptide shown in FIG. 2 (SEQ ID NO:2). FGF-19 variant polypeptides may be those that are encoded by a FGF-19 variant polynucleotide.

The term “positives”, in the context of the amino acid sequence identity comparisons performed as described above, includes amino acid residues in the sequences compared that are not only identical, but also those that have similar properties. Amino acid residues that score a positive value to an amino acid residue of interest are those that are either identical to the amino acid residue of interest or are a preferred substitution (as defined in Table 6 below) of the amino acid residue of interest.

For purposes herein, the % value of positives of a given amino acid sequence A to, with, or against a given amino acid sequence B (which can alternatively be phrased as a given amino acid sequence A that has or comprises a certain % positives to, with, or against a given amino acid sequence B) is calculated as follows:

100 times the fraction X/Y

where X is the number of amino acid residues scoring a positive value as defined above by the sequence alignment program ALIGN-2 in that program's alignment of A and B, and where Y is the total number of amino acid residues in B. It will be appreciated that where the length of amino acid sequence A is not equal to the length of amino acid sequence B, the % positives of A to B will not equal the % positives of B to A.

“Isolated,” when used to describe the various polypeptides disclosed herein, means polypeptide that has been identified and separated and/or recovered from a component of its natural environment. Preferably, the isolated polypeptide is free of association with all components with which it is naturally associated. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the FGF-19 natural environment will not be present. Ordinarily, however, isolated polypeptide will be prepared by at least one purification step.

An “isolated” nucleic acid molecule encoding a FGF-19 polypeptide is a nucleic acid molecule that is identified and separated from at least one contaminant nucleic acid molecule with which it is ordinarily associated in the natural source of the FGF-19-encoding nucleic acid. Preferably, the isolated nucleic is free of association with all components with which it is naturally associated. An isolated FGF-19-encoding nucleic acid molecule is other than in the form or setting in which it is found in nature. Isolated nucleic acid molecules therefore are distinguished from the FGF-19-encoding nucleic acid molecule as it exists in natural cells. However, an isolated nucleic acid molecule encoding a FGF-19 polypeptide includes FGF-19-encoding nucleic acid molecules contained in cells that ordinarily express FGF-19 where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-FGF-19 monoclonal antibodies (including agonist, antagonist, and neutralizing antibodies), anti-FGF-19 antibody compositions with polyepitopic specificity, single chain anti-FGF-19 antibodies, and fragments of anti-FGF-19 antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a FGF-19 polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulinconstant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of FGF-19 which retain a biological and/or an immunological activity of native or naturally-occurring FGF-19, wherein “biological” activity refers to a biological function (either inhibitory or stimulatory) caused by a native or naturally-occurring FGF-19 other than the ability to induce the production of an antibody against an antigenic epitope possessed by a native or naturally-occurring FGF-19 and an “immunological” activity refers to the ability to induce the production of an antibody against an antigenic epitope possessed by a native or naturally-occurring FGF-19. A preferred biological activity includes any one or more of the following activities: increases metabolism (or metabolic rate) in an individual, decreases body weight of an individual, decreases adiposity in an individual, decreases glucose uptake into adipocytes, increases leptin release from adipocytes, decreases triglycerides in an individual, and decreases free fatty acids in an individual. It is understood that some of the activities of FGF-19 are directly induced by FGF-19 and some are indirectly induced, however, each are the result of the presence of FGF-19 and would not otherwise have the result in the absence of FGF-19.

The term “antagonist” is used in the broadest sense, and includes any molecule that partially or fully blocks, inhibits, or neutralizes a biological activity of a native FGF-19 polypeptide disclosed herein. In a similar manner, the term “agonist” is used in the broadest sense and includes any molecule that mimics a biological activity of a native FGF-19 polypeptide disclosed herein. Suitable agonist or antagonist molecules specifically include agonist or antagonist antibodies or antibody fragments, fragments or amino acid sequence variants of native FGF-19 polypeptides, peptides, small organic molecules, etc. Methods for identifying agonists or antagonists of a FGF-19 polypeptide may comprise contacting a FGF-19 polypeptide with a candidate agonist or antagonist molecule and measuring a detectable change in one or more biological activities normally associated with the FGF-19 polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

“Individual” is any subject, preferably a mammal, more preferably a human.

“Obesity” refers to a condition whereby a mammal has a Body Mass Index (BMI), which is calculated as weight (kg) per height² (meters), of at least 25.9. Conventionally, those persons with normal weight have a BMI of 19.9 to less than 25.9. The obesity herein may be due to any cause, whether genetic or environmental. Examples of disorders that may result in obesity or be the cause of obesity include overeating and bulimia, polycystic ovarian disease, craniopharyngioma, the Prader-Willi Syndrome, Frohlich's syndrome, Type II diabetes, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia.

“Conditions related to obesity” refer to conditions which are the result of or which are exasperated by obesity, such as, but not limited to dermatological disorders such as infections, varicose veins, Acanthosis nigricans, and eczema, exercise intolerance, diabetes mellitus, insulin resistance, hypertension, hypercholesterolemia, cholelithiasis, osteoarthritis, orthopedic injury, thromboembolic disease, cancer, and coronary (or cardiovascular) heart disease, particular those cardiovascular conditions associated with high triglycerides and free fatty acids in an individual.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′₂,) and Fv fragments; diabodies; linear antibodies (Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′₂) fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a FGF-19 polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

TABLE 2

| | | |
|---|---|---|
| PRO | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 15 = 33.3%

TABLE 3

| | | |
|---|---|---|
| PRO | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 10 = 50%

TABLE 4

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 6 divided by 14 = 42.9%

TABLE 5

| | | |
|---|---|---|
| PRO-DNA | NNNNNNNNNNNN | (Length = 12 nucleotides) |
| Comparison DNA | NNNNLLLVV | (Length = 9 nucleotides) |

% nucleic acid sequence identity = (the number of identically matching nucleotides between the two nucleic acid sequences as determined by ALIGN-2) divided by (the total number of nucleotides of the PRO-DNA nucleic acid sequence) = 4 divided by 12 = 33.3%

II. Compositions and Methods of the Invention

A. Full-Length FGF-19 Polypeptide

The present invention provides newly identified and isolated nucleotide sequences encoding polypeptides referred to in the present application as FGF-19 (or also UNQ334). In particular, cDNA encoding a FGF-19 polypeptide has been identified and isolated, as disclosed in further detail in the Examples below. It is noted that proteins produced in separate expression rounds may be given different PRO numbers but the UNQ number is unique for any given DNA and the encoded protein, and will not be changed. However, for sake of simplicity, in the present specification the protein encoded by DNA49435-1219 as well as all further native homologues and variants included in the foregoing definition of FGF-19 (also sometimes referred to as PRO533), will be referred to as “FGF-19”, regardless of their origin or mode of preparation.

As disclosed in the Examples below, a cDNA clone designated herein as DNA49435-1219 has been deposited with the ATCC. The actual nucleotide sequence of the clone can readily be determined by the skilled artisan by sequencing of the deposited clone using routine methods in the art. The predicted amino acid sequence can be determined from the nucleotide sequence using routine skill. For the FGF-19 polypeptide and encoding nucleic acid described herein, Applicants have identified what is believed to be the reading frame best identifiable with the sequence information available at the time.

Using the ALIGN-2 sequence alignment computer program referenced above, it has been found that the full-length native sequence FGF-19 (shown in FIG. 2 and SEQ ID NO:2) has certain amino acid sequence identity with AF007268_{—}1. Accordingly, it is presently believed that the FGF-19 polypeptide disclosed in the present application is a newly identified member of the fibroblast growth factor protein family and may possess one or more biological and/or immunological activities or properties typical of that protein family.

B. FGF-19 Variants

In addition to the full-length native sequence FGF-19 polypeptides described herein, it is contemplated that FGF-19 variants can be prepared. FGF-19 variants can be prepared by introducing appropriate nucleotide changes into the FGF-19 DNA, and/or by synthesis of the desired FGF-19 polypeptide. Those skilled in the art will appreciate that amino acid changes may alter post-translational processes of the FGF-19, such as changing the number or position of glycosylation sites or altering the membrane anchoring characteristics.

Variations in the native full-length sequence FGF-19 or in various domains of the FGF-19 described herein, can be made, for example, using any of the techniques and guidelines for conservative and non-conservative mutations set forth, for instance, in U.S. Pat. No. 5,364,934. Variations may be a substitution, deletion or insertion of one or more codons encoding the FGF-19 that results in a change in the amino acid sequence of the FGF-19 as compared with the native sequence FGF-19. Optionally the variation is by substitution of at least one amino acid with any other amino acid in one or more of the domains of the FGF-19. Guidance in determining which amino acid residue may be inserted, substituted or deleted without adversely affecting the desired activity may be found by comparing the sequence of the FGF-19 with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

FGF-19 polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the FGF-19 polypeptide.

FGF-19 fragments may be prepared by any of a number of conventional techniques. Desired peptide fragments may be chemically synthesized. An alternative approach involves generating FGF-19 fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, FGF-19 polypeptide fragments share at least one biological and/or immunological activity with the native FGF-19 polypeptide shown in FIG. 2 (SEQ ID NO:2).

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the FGF-19 polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res. 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the FGF-19 variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

C. Modifications of FGF-19

Covalent modifications of FGF-19 are included within the scope of this invention. One type of covalent modification includes reacting targeted amino acid residues of a FGF-19 polypeptide with an organic derivatizing agent that is capable of reacting with selected side chains or the - or C-terminal residues of the FGF-19. Derivatization with bifunctional agents is useful, for instance, for crosslinking FGF-19 to a water-insoluble support matrix or surface for use in the method for purifying anti-FGF-19 antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleinides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the FGF-19 polypeptide included within the scope of this invention comprises altering the native glycosylation pattern of the polypeptide. “Altering the native glycosylation pattern” is intended for purposes herein to mean deleting one or more carbohydrate moieties found in native sequence FGF-19 (either by removing the underlying glycosylation site or by deleting the glycosylation by chemical and/or enzymatic means), and/or adding one or more glycosylation sites that are not present in the native sequence FGF-19. In addition, the phrase includes qualitative changes in the glycosylation of the native proteins, involving a change in the nature and proportions of the various carbohydrate moieties present.

Addition of glycosylation sites to the FGF-19 polypeptide may be accomplished by altering the amino acid sequence. The alteration may be made, for example, by the addition of, or substitution by, one or more serine or threonine residues to the native sequence FGF-19 (for O-linked glycosylation sites). The FGF-19 amino acid sequence may optionally be altered through changes at the DNA level, particularly by mutating the DNA encoding the FGF-19 polypeptide at preselected bases such that codons are generated that will translate into the desired amino acids.

Another means of increasing the number of carbohydrate moieties on the FGF-19 polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the FGF-19 polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzmmol., 138:350 (1987).

Another type of covalent modification of FGF-19 comprises linking the FGF-19 polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The FGF-19 of the present invention may also be modified in a way to form a chimeric molecule comprising FGF-19 fused to another, heterologous polypeptide or amino acid sequence.

In one embodiment, such a chimeric molecule comprises a fusion of the FGF-19 with a tag polypeptide which provides an epitope to which an anti-tag antibody can selectively bind. The epitope tag is generally placed at the amino- or carboxyl-terminus of the FGF-19. The presence of such epitope-tagged forms of the FGF-19 can be detected using an antibody against the tag polypeptide. Also, provision of the epitope tag enables the FGF-19 to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 31(6):547-553 (1990)]. Other tag polypeptides include the Flag-peptide [Hopp et al., BioTechnology, 6:1204-1210 (1988)]; the KT3 epitope peptide [Martinet al., Science, 255:192-194 (1992)]; an α-tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the FGF-19 with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a FGF-19 polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

D. Preparation of FGF-19

The description below relates primarily to production of FGF-19 by culturing cells transformed or transfected with a vector containing FGF-19 nucleic acid. It is, of course, contemplated that alternative methods, which are well known in the art, may be employed to prepare FGF-19. For instance, the FGF-19 sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the FGF-19 may be chemically synthesized separately and combined using chemical or enzymatic methods to produce the full-length FGF-19.

1. Isolation of DNA Encoding FGF-19

DNA encoding FGF-19 may be obtained from a cDNA library prepared from tissue believed to possess the FGF-19 mRNA and to express it at a detectable level. Accordingly, human FGF-19 DNA can be conveniently obtained from a cDNA library prepared from human tissue, such as described in the Examples. The FGF-19-encoding gene may also be obtained from a genomic library or by known synthetic procedures (e.g., automated nucleic acid synthesis).

Libraries can be screened with probes (such as antibodies to the FGF-19 or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding FGF-19 is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for FGF-19 production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537(1990) and Mansour et al., Nature 336:348-352(1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for FGF-19-encoding vectors. Saccharomyces cerevisiae is a commonly used lower eukaryotic host microorganism. Others include Schizosaccharomyces pombe (Beach and Nurse, Nature, 290:140[1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichodenna reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated FGF-19 are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR(CHO, Urlaub and Chasin, Proc. Natl. Acad. Sci. USA, 77:4216(1980)); mouse sertoli cells (TM4, Mather, Biol. Reprod., 23:243-251 (1980)); human lung cells (W 138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding FGF-19 may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The FGF-19 may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the FGF-19-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 21 plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the FGF-19-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene 7:141 (1979); Tschemper et al., Gene 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the FGF-19-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature. 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding FGF-19.

Examples of suitable promoting sequences for use with yeast hosts include the promoters for 3-phosphoglycerate kinase [Hitzeman et al., J. Biol. Chem. 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

FGF-19 transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the FGF-19 by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the FGF-19 coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding FGF-19.

Still other methods, vectors, and host cells suitable for adaptation to the synthesis of FGF-19 in recombinant vertebrate cell culture are described in Gething et al., Nature, 293:620-625 (1981); Mantei et al., Nature, 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence FGF-19 polypeptide or against a synthetic peptide based on the DNA sequences provided herein or against exogenous sequence fused to FGF-19 DNA and encoding a specific antibody epitope.

5. Purification of Polypeptide

Forms of FGF-19 may be recovered from culture medium or from host cell lysates. If membrane-bound, it can be released from the membrane using a suitable detergent solution (e.g. Triton-X 100) or by enzymatic cleavage. Cells employed in expression of FGF-19 can be disrupted by various physical or chemical means, such as freeze-thaw cycling, sonication, mechanical disruption, or cell lysing agents.

It may be desired to purify FGF-19 from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the FGF-19. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular FGF-19 produced.

E. Uses for FGF-19

Nucleotide sequences (or their complement) encoding FGF-19 have various applications in the art of molecular biology, including uses as hybridization probes, in chromosome and gene mapping and in the generation of anti-sense RNA and DNA. FGF-19 nucleic acid will also be useful for the preparation of FGF-19 polypeptides by the recombinant techniques described herein.

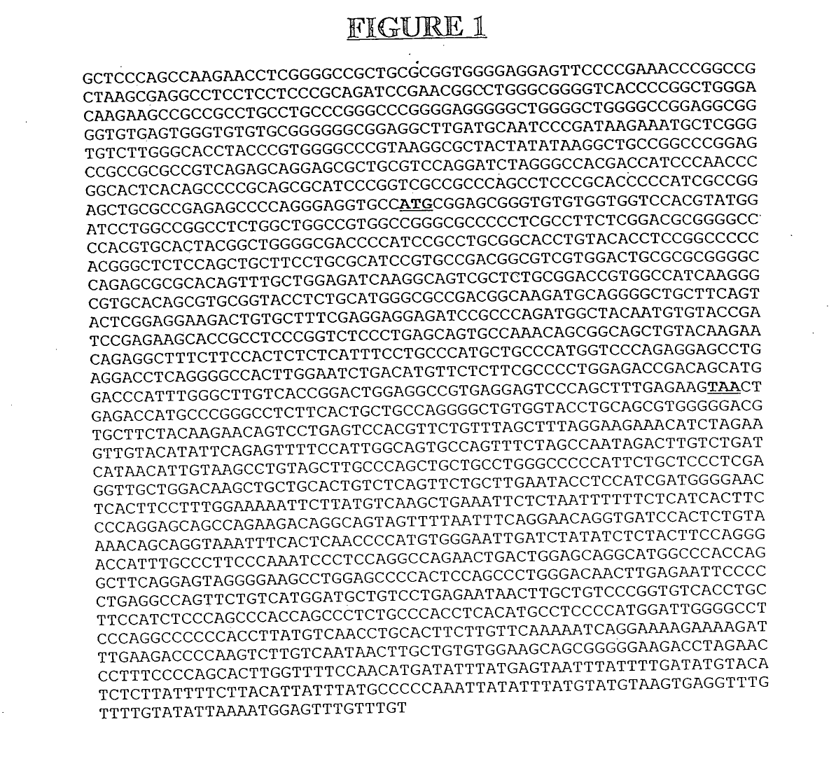

The full-length native sequence FGF-19 gene (SEQ ID NO:1), or portions thereof, may be used as hybridization probes for a cDNA library to isolate the full-length FGF-19 cDNA or to isolate still other cDNAs (for instance, those encoding naturally-occurring variants of FGF-19 or FGF-19 from other species) which have a desired sequence identity to the FGF-19 sequence disclosed in FIG. 1 (SEQ ID NO:1). Optionally, the length of the probes will be about 20 to about 50 bases. The hybridization probes may be derived from at least partially novel regions of the nucleotide sequence of SEQ ID NO:1 wherein those regions may be determined without undue experimentation or from genomic sequences including promoters, enhancer elements and introns of native sequence FGF-19. By way of example, a screening method will comprise isolating the coding region of the FGF-19 gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the FGF-19 gene of the present invention can be used to screen libraries of human cDNA, genomic DNA or mRNA to determine which members of such libraries the probe hybridizes to. Hybridization techniques are described in further detail in the Examples below.

Any EST sequences disclosed in the present application may similarly be employed as probes, using the methods disclosed herein.

Other useful fragments of the FGF-19 nucleic acids include antisense or sense oligonucleotides comprising a singe-stranded nucleic acid sequence (either RNA or DNA) capable of binding to target FGF-19 mRNA (sense) or FGF-19 DNA (antisense) sequences. Antisense or sense oligonucleotides, according to the present invention, comprise a fragment of the coding region of FGF-19 DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to block expression of FGF-19 proteins. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

The probes may also be employed in PCR techniques to generate a pool of sequences for identification of closely related FGF-19 coding sequences.

Nucleotide sequences encoding a FGF-19 can also be used to construct hybridization probes for mapping the gene which encodes that FGF-19 and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for FGF-19 encode a protein which binds to another protein (example, where the FGF-19 is a receptor), the FGF-19 can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor FGF-19 can be used to isolate correlative ligand(s). Screening assays can be designed to find lead compounds that mimic the biological activity of a native FGF-19 or a receptor for FGF-19. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

Nucleic acids which encode FGF-19 or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding FGF-19 can be used to clone genomic DNA encoding FGF-19 in accordance with established techniques and the genomic sequences used to generate transgenic animals that contain cells which express DNA encoding FGF-19. Methods for generating transgenic animals, particularly animals such as mice or rats, have become conventional in the art and are described, for example, in U.S. Pat. Nos. 4,736,866 and 4,870,009. Typically, particular cells would be targeted for FGF-19 transgene incorporation with tissue-specific enhancers. Transgenic animals that include a copy of a transgene encoding FGF-19 introduced into the germ line of the animal at an embryonic stage can be used to examine the effect of increased expression of DNA encoding FGF-19. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of FGF-19 can be used to construct a FGF-19 “knock out” animal which has a defective or altered gene encoding FGF-19 as a result of homologous recombination between the endogenous gene encoding FGF-19 and altered genomic DNA encoding FGF-19 introduced into an embryonic stem cell of the animal. For example, cDNA encoding FGF-19 can be used to clone genomic DNA encoding FGF-19 in accordance with established techniques. A portion of the genomic DNA encoding FGF-19 can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the FGF-19 polypeptide.

Nucleic acid encoding the FGF-19 polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The FGF-19 polypeptides described herein may also be employed as molecular weight markers for protein electrophoresis purposes.

The nucleic acid molecules encoding the FGF-19 polypeptides or fragments thereof described herein are useful for chromosome identification. In this regard, there exists an ongoing need to identify new chromosome markers, since relatively few chromosome marking reagents, based upon actual sequence data are presently available. Each FGF-19 nucleic acid molecule of the present invention can be used as a chromosome marker.

The FGF-19 polypeptides and nucleic acid molecules of the present invention may also be used for tissue typing, wherein the FGF-19 polypeptides of the present invention may be differentially expressed in one tissue as compared to another. FGF-19 nucleic acid molecules will find use for generating probes for PCR, Northern analysis, Southern analysis and Western analysis.

The FGF-19 polypeptides and modulators thereof described herein may also be employed as therapeutic agents. The FGF-19 polypeptides and modulators thereof of the present invention can be formulated according to known methods to prepare pharmaceutically useful compositions, whereby the FGF-19 product hereof is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

The route of administration is in accord with known methods, e.g. injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a FGF-19 polypeptide or agonist or antagonist thereof is employed, normal dosage amounts may vary from about 10 ng/kg to up to 100 mg/kg of mammal body weight or more per day, preferably about 1 g/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of a FGF-19 polypeptide or modulator is desired in a formulation with release characteristics suitable for the treatment of any disease or disorder requiring administration of the FGF-19 polypeptide or modulator, microencapsulation is contemplated. Microencapsulation of recombinant proteins for sustained release has been successfully performed with human growth hormone (rhGH), interferon- (rhIFN-), interleukin-2, and MN rgp120. Johnson et al., Nat. Med. 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

The therapeutic agents and compositions comprising FGF-19 provided herein can be used in a number of applications. The applications include treating an individual with obesity or a condition associated with obesity. In one aspect, FGF-19 is administered to an individual in need thereof in an amount effective to treat the condition. Preferably, the condition is one which requires at least one of the following to be treated: an increase in metabolism, a decrease in body weight, a decrease in body fat, a decrease in triglycerides, a decrease in free fatty acids, an increase in glucose release from adipocytes, an increase in insulin sensitivity and/or an increase in leptin release from adipocytes. Each of these parameters can be measured by standard methods, for example, by measuring oxygen consumption to determine metabolic rate, using scales to determine weight, and measuring size to determine fat. Moreover, the presence and amount of triglycerides, free fatty acids, glucose and leptin can be determined by standard methods. Each of these parameters is exemplified below in the specific examples.

FGF-19 and compositions comprising FGF-19 are preferably used in vivo. However, as discussed below, administration can be in vitro such as in the methods described below for screening for modulators of FGF-19. Although, it is understood that modulators of FGF-19 can also be identified by the use of animal models and samples from patients.

This invention encompasses methods of screening compounds to identify those that mimic or enhance the FGF-19 polypeptide (agonists) or prevent or inhibit the effect of the FGF-19 polypeptide (antagonists). Agonists and antagonists are referred to as modulators herein. Screening assays for antagonist drug candidates are designed to identify compounds that bind or complex with the FGF-19 polypeptides encoded by the genes identified herein, or otherwise interfere with the interaction of the encoded polypeptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a FGF-19 polypeptide encoded by a nucleic acid identified herein under conditions and for a time sufficient to allow these two components to interact.

In binding assays, the interaction is binding and the complex formed can be isolated or detected in the reaction mixture. In a particular embodiment, the FGF-19 polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the FGF-19 polypeptide and drying. Alternatively, an immobilized antibody, e.g., a monoclonal antibody, specific for the FGF-19 polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular FGF-19 polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a FGF-19 polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the FGF-19 polypeptide may be added to a cell along with the compound to be screened for a particular activity and the ability of the compound to inhibit the activity of interest in the presence of the FGF-19 polypeptide indicates that the compound is an antagonist to the FGF-19 polypeptide. Alternatively, antagonists may be detected by combining the FGF-19 polypeptide and a potential antagonist with membrane-bound FGF-19 polypeptide receptors or recombinant receptors under appropriate conditions for a competitive inhibition assay. The FGF-19 polypeptide can be labeled, such as by radioactivity, such that the number of FGF-19 polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the FGF-19 polypeptide and a cDNA library created from this RNA is divided into pools and used to transfect COS cells or other cells that are not responsive to the FGF-19 polypeptide. Transfected cells that are grown on glass slides are exposed to labeled FGF-19 polypeptide. The FGF-19 polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled FGF-19 polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled FGF-19 polypeptide in the presence of the candidate compound. The ability of the compound to enhance or block this interaction could then be measured.

More specific examples of potential antagonists include an oligonucleotide that binds to the fusions of immunoglobulin with FGF-19 polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the FGF-19 polypeptide that recognizes the receptor but imparts no effect, thereby competitively inhibiting the action of the FGF-19 polypeptide.

In one embodiment herein where competitive binding assays are performed, FGF receptor 4 or an antibody to FGF-19 is used as a competitor.

Another potential FGF-19 polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature FGF-19 polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res. 6:3073 (1979); Cooney et al., Science, 241: 456 (1988); Dervan et al., Science, 251:1360 (1991)), thereby preventing transcription and the production of the FGF-19 polypeptide. The antisense RNA oligonucleotide hybridizes to the mRNA in vivo and blocks translation of the mRNA molecule into the FGF-19 polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the FGF-19 polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the FGF-19 polypeptide, thereby blocking the normal biological activity of the FGF-19 polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

It is appreciated that all the assays provided herein can be used to screen a wide variety of candidate bioactive agents. The term “candidate bioactive agent”, “candidate agent” or “drug candidate” or grammatical equivalents as used herein describes any molecule, e.g., protein, oligopeptide, small organic molecule, polysaccharide, polynucleotide, purine analog, etc., to be tested for bioactive agents that are capable of directly or indirectly altering either the cellular activity phenotype or the expression of a FGF-19 sequence, including both nucleic acid sequences and protein sequences.

Candidate agents can encompass numerous chemical classes, though typically they are organic molecules, preferably small organic compounds having a molecular weight of more than 100 and less than about 2,500 daltons (d). Small molecules are further defined herein as having a molecular weight of between 50 d and 2000 d. In another embodiment, small molecules have a molecular weight of less than 1500, or less than 1200, or less than 1000, or less than 750, or less than 500 d. In one embodiment, a small molecule as used herein has a molecular weight of about 100 to 200 d. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes amino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the methods of the invention. Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10): 1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

As described above generally for proteins, nucleic acid candidate bioactive agents may be naturally occurring nucleic acids, random nucleic acids, or “biased” random nucleic acids. For example, digests of prokaryotic or eukaryotic genomes may be used as is outlined above for proteins.

In a preferred embodiment, the candidate bioactive agents are organic chemical moieties, a wide variety of which are available in the literature.

In a preferred embodiment, as outlined above, screens may be done on individual genes and gene products (proteins). In a preferred embodiment, the gene or protein has been identified as described below in the Examples as a differentially expressed gene associated with particular tissues and thus conditions related to those tissues. Thus, in one embodiment, screens are designed to first find candidate agents that can bind to FGF-19, and then these agents may be used in assays that evaluate the ability of the candidate agent to modulate FGF-19 activity. Thus, as will be appreciated by those in the art, there are a number of different assays which may be run.

Screening for agents that modulate the activity of FGF-19 may also be done. In a preferred embodiment, methods for screening for a bioactive agent capable of modulating the activity of FGF-19 comprise the steps of adding a candidate bioactive agent to a sample of FGF-19 and determining an alteration in the biological activity of FGF-19. “Modulating the activity of FGF-19” includes an increase in activity, a decrease in activity, or a change in the type or kind of activity present. Thus, in this embodiment, the candidate agent should both bind to FGF-19 (although this may not be necessary), and alter its biological or biochemical activity as defined herein. The methods include both in vitro screening methods, as are generally outlined above, and in vivo screening of cells for alterations in the presence, expression, distribution, activity or amount of FGF-19.

Thus, in this embodiment, the methods comprise combining a sample and a candidate bioactive agent, and evaluating the effect on FGF-19 activity. By “FGF-19 protein activity” or grammatical equivalents herein is meant at least one of the FGF-19 protein's biological activities as described above.

In a preferred embodiment, the activity of the FGF-19 protein is increased; in another preferred embodiment, the activity of the FGF-19 protein is decreased. Thus, bioactive agents that are antagonists are preferred in some embodiments, and bioactive agents that are agonists may be preferred in other embodiments.

In one aspect of the invention, cells containing FGF-19 sequences are used in drug screening assays by evaluating the effect of drug candidates on FGF-19. Cell type include normal cells, tumor cells, and adipocytes.

Methods of assessing FGF-19 activity such as changes in glucose uptake, leptin release, metabolism, triglyceride and free fatty acid levels, body weight and body fat, are known in the art and are exemplified below in the examples.

In a preferred embodiment, the methods comprise adding a candidate bioactive agent, as defined above, to a cell comprising FGF-19. Preferred cell types include almost any cell. The cells contain a nucleic acid, preferably recombinant, that encodes a FGF-19 protein. In a preferred embodiment, a library of candidate agents are tested on a plurality of cells.

In one aspect, the assays are evaluated in the presence or absence or previous or subsequent exposure to physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

The FGF-19 sequences provided herein can also be used in methods of diagnosis. Overexpression of FGF-19 may indicate an abnormally high metabolic rate and underexpression may indicate a propensity for obesity and related disorders. Moreover, a sample from a patient may be analyzed for mutated or disfunctional FGF-19. Generally, such methods include comparing a sample from a patient and comparing FGF-19 expression to that of a control.

F. Anti-FGF-19 Antibodies

The present invention further provides anti-FGF-19 antibodies. Exemplary antibodies include polyclonal, monoclonal, humanized, bispecific, and heteroconjugate antibodies.

1. Polyclonal Antibodies

The anti-FGF-19 antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the FGF-19 polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-FGF-19 antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the FGF-19 polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against FGF-19. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem. 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Human and Humanized Antibodies

The anti-FGF-19 antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol. 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783(1992); Lonberg et al., Nature 368 856-859(1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

4. Bispecific Antibodies

Bispecific antibodies are monoclonal, preferably human or humanized, antibodies that have binding specificities for at least two different antigens. In the present case, one of the binding specificities is for the FGF-19, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies may bind to two different epitopes on a given FGF-19 polypeptide herein. Alternatively, an anti-FGF-19 polypeptide arm may be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular FGF-19 polypeptide. Bispecific antibodies may also be used to localize cytotoxic agents to cells which express a particular FGF-19 polypeptide. These antibodies possess a FGF-19-binding arm and an arm which binds a cytotoxic agent or a radionuclide chelator, such as EOTUBE, DPTA, DOTA, or TETA. Another bispecific antibody of interest binds the FGF-19 polypeptide and further binds tissue factor (TF).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

6. Effector Function Engineering

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al. Cancer Research, 53: 2560-2565 (1993). Alternatively, an antibody can be engineered that has dual Fc regions and may thereby have enhanced complement lysis and ADCC capabilities. See Stevenson et al., Anti-Cancer Drug Design. 3: 219-230 (1989).

7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³³I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

8. Immunoliposomes

The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

9. Pharmaceutical Compositions of Antibodies

Antibodies specifically binding a FGF-19 polypeptide identified herein, as well as other molecules identified by the screening assays disclosed hereinbefore, can be administered for the treatment of various disorders in the form of pharmaceutical compositions.

If the FGF-19 polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulflhydryl residues, lyophilizing from acidic solutions, controlling moisture content, using appropriate additives, and developing specific polymer matrix compositions.

G. Uses for Anti-FGF-19 Antibodies

The anti-FGF-19 antibodies of the invention have various utilities. For example, anti-FGF-19 antibodies may be used in diagnostic assays for FGF-19, e.g., detecting its expression in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as 3H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth. 40:219 (1981); and Nygren, J. Histochem. and Cytochem., 30:407 (1982).

Anti-FGF-19 antibodies also are useful for the affinity purification of FGF-19 from recombinant cell culture or natural sources. In this process, the antibodies against FGF-19 are immobilized on a suitable support, such a Sephadex resin or filter paper, using methods well known in the art. The immobilized antibody then is contacted with a sample containing the FGF-19 to be purified, and thereafter the support is washed with a suitable solvent that will remove substantially all the material in the sample except the FGF-19, which is bound to the immobilized antibody. Finally, the support is washed with another suitable solvent that will release the FGF-19 from the antibody.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Isolation of cDNA Clones Encoding a Human FGF-19

The EST sequence accession number AF007268, a murine fibroblast growth factor (FGF-15) was used to search various public EST databases (e.g., GenBank, Dayhoff, etc.). The search was performed using the computer program BLAST or BLAST2 [Altschul et al., Methods in Enzymology, 266:460480 (1996)] as a comparison of the ECD protein sequences to a 6 frame translation of the EST sequences. The search resulted in a hit with GenBank EST AA220994, which has been identified as STRATAGENE NT2 neuronal precursor 937230. The sequence of AA220994 is also referred to herein as DNA47412.

Based on the DNA47412 sequence, oligonucleotides were synthesized: 1) to identify by PCR a cDNA library that contained the sequence of interest, and 2) for use as probes to isolate a clone of the full-length coding sequence for FGF-19. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

| | | |
|---|---|---|
| forward PCR primer | 5′-ATCCGCCCAGATGGCTACAATGTGTA-3′, | (SEQ ID NO:3) |
| and | | |
| reverse PCR primer | 5′-CCAGTCCGGTGACAAGCCCAAA-3′. | (SEQ ID NO:4) |

Additionally, a synthetic oligonucleotide hybridization probe was constructed from the DNA47412 sequence which had the following nucleotide sequence:

Hybridization Probe

5′-GCCTCCCGGTCTCCCTGAGCAGTGCCAAACAGCGGCAGTGTA-3′ (SEQ ID NO:5).

RNA for construction of the cDNA libraries was isolated from human fetal retina tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

DNA sequencing of the clones isolated as described above gave the full-length DNA sequence for a full-length FGF-19 polypeptide (designated herein as DNA49435-1219 [FIG. 1, SEQ ID NO:1]) and the derived protein sequence for that FGF-19 polypeptide.

The full length clone identified above contained a single open reading frame with an apparent translational initiation site at nucleotide positions 464466 and a stop signal at nucleotide positions 1112-1114 (FIG. 1, SEQ ID NO:1). The predicted polypeptide precursor is 216 amino acids long, has a calculated molecular weight of approximately 24,003 daltons and an estimated pI of approximately 6.99. Analysis of the full-length FGF-19 sequence shown in FIG. 2 (SEQ ID NO:2) evidences the presence of a variety of important polypeptide domains as shown in FIG. 2, wherein the locations given for those important polypeptide domains are approximate as described above. Chromosome mapping evidences that the FGF-19-encoding nucleic acid maps to chromosome 11q13.1, band q13.1, in humans. Clone DNA49435-1219 has been deposited with ATCC on Nov. 21, 1997 and is assigned ATCC deposit no. 209480.

An analysis of the Dayhoff database (version 35.45 SwissProt 35), using the ALIGN-2 sequence alignment analysis of the full-length sequence shown in FIG. 2 (SEQ ID NO:2), evidenced sequence identity between the FGF-19 amino acid sequence and the following Dayhoff sequences: AF007268_{—}1, S54407, P_W52596, FGF2_XENLA, P_W53793, AB002097_{—}1, P_R27966, HSU67918_{—}1, S23595, and P_R70824.

### Example 2

### Use of FGF-19 as a Hybridization Probe

The following method describes use of a nucleotide sequence encoding FGF-19 as a hybridization probe.

DNA comprising the coding sequence of full-length or mature FGF-19 is employed as a probe to screen for homologous DNAs (such as those encoding naturally-occurring variants of FGF-19) in human tissue cDNA libraries or human tissue genomic libraries.

Hybridization and washing of filters containing either library DNAs is performed under the following high stringency conditions. Hybridization of radiolabeled FGF-19-derived probe to the filters is performed in a solution of 50% formamide, 5×SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence FGF-19 can then be identified using standard techniques known in the art.

### Example 3

### Expression of FGF-19 in E. coli

This example illustrates preparation of an unglycosylated form of FGF-19 by recombinant expression in E. coli.

The DNA sequence encoding FGF-19 is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the FGF-19 coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized FGF-19 protein can then be purified using a metal chelating column under conditions that allow tight binding of the protein.

FGF-19 may be expressed in E. coli in a poly-His tagged form, using the following procedure. The DNA encoding FGF-19 is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) lon galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate.2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded FGF-19 polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### Example 4

### Expression of FGF-19 in Mammalian Cells

This example illustrates preparation of a potentially glycosylated form of FGF-19 by recombinant expression in mammalian cells.

The vector, pRK5 (see EP 307,247, published Mar. 15, 1989), is employed as the expression vector. Optionally, the FGF-19 DNA is ligated into pRK5 with selected restriction enzymes to allow insertion of the FGF-19 DNA using ligation methods such as described in Sambrook et al., supra. The resulting vector is called pRK5-FGF-19.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-FGF-19 DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of FGF-19 polypeptide. The cultures containing transfected cells may undergo further incubation (in serum free medium) and the medium is tested in selected bioassays.

In an alternative technique, FGF-19 may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-FGF-19 DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed FGF-19 can then be concentrated and purified by any selected method, such as dialysis and/or column chromatography.

In another embodiment, FGF-19 can be expressed in CHO cells. The pRK5-FGF-19 can be transfected into CHO cells using known reagents such as CaPO₄ or DEAE-dextran. As described above, the cell cultures can be incubated, and the medium replaced with culture medium (alone) or medium containing a radiolabel such as 35S-methionine. After determining the presence of FGF-19 polypeptide, the culture medium may be replaced with serum free medium. Preferably, the cultures are incubated for about 6 days, and then the conditioned medium is harvested. The medium containing the expressed FGF-19 can then be concentrated and purified by any selected method.

Epitope-tagged FGF-19 may also be expressed in host CHO cells. The FGF-19 may be subcloned out of the pRK5 vector. The subclone insert can undergo PCR to fuse in frame with a selected epitope tag such as a poly-his tag into a Baculovirus expression vector. The poly-his tagged FGF-19 insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged FGF-19 can then be concentrated and purified by any selected method, such as by Ni²⁺-chelate affinity chromatography.

FGF-19 may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁻⁷ cells are frozen in an ampule for further growth and production as described below.

The ampules containing the plasmid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3L production spinner is seeded at 1.2×10⁶ cells/mL. On day 0, the cell number pH is determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

### Example 5

### Expression of FGF-19 in Yeast

The following method describes recombinant expression of FGF-19 in yeast.

First, yeast expression vectors are constructed for intracellular production or secretion of FGF-19 from the ADH2/GAPDH promoter. DNA encoding FGF-19 and the promoter is inserted into suitable restriction enzyme sites in the selected plasmid to direct intracellular expression of FGF-19. For secretion, DNA encoding FGF-19 can be cloned into the selected plasmid, together with DNA encoding the ADH2/GAPDH promoter, a native FGF-19 signal peptide or other mammalian signal peptide, or, for example, a yeast alpha-factor or invertase secretory signal/leader sequence, and linker sequences (if needed) for expression of FGF-19.

Yeast cells, such as yeast strain AB 110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant FGF-19 can subsequently be isolated and purified by removing the yeast cells from the fermentation medium by centrifugation and then concentrating the medium using selected cartridge filters. The concentrate containing FGF-19 may further be purified using selected column chromatography resins.

### Example 6

### Expression of FGF-19 in Baculovirus-Infected Insect Cells

The following method describes recombinant expression of FGF-19 in Baculovirus-infected insect cells.

The sequence coding for FGF-19 is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding FGF-19 or the desired portion of the coding sequence of FGF-19 such as the sequence encoding the extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged FGF-19 can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged FGF-19 are pooled and dialyzed against loading buffer.

Alternatively, purification of the IgG tagged (or Fc tagged) FGF-19 can be performed using known chromatography techniques, including for instance, Protein A or protein G column chromatography.

### Example 7

### Preparation of Antibodies that Bind FGF-19

This example illustrates preparation of monoclonal antibodies which can specifically bind FGF-19. Techniques for producing the monoclonal antibodies are known in the art and are described, for instance, in Goding, supra. Immunogens that may be employed include purified FGF-19, fusion proteins containing FGF-19, and cells expressing recombinant FGF-19 on the cell surface. Selection of the immunogen can be made by the skilled artisan without undue experimentation.

Mice, such as Balb/c, are immunized with the FGF-19 immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-FGF-19 antibodies.

After a suitable antibody titer has been detected, the animals “positive” for antibodies can be injected with a final intravenous injection of FGF-19. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against FGF-19. Determination of “positive” hybridoma cells secreting the desired monoclonal antibodies against FGF-19 is within the skill in the art.

The positive hybridoma cells can be injected intraperitoneally into syngeneic Balb/c mice to produce ascites containing the anti-FGF-19 monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 8

### Purification of FGF-19 Polypeptides Using Specific Antibodies

Native or recombinant FGF-19 polypeptides may be purified by a variety of standard techniques in the art of protein purification. For example, pro-FGF-19 polypeptide, mature FGF-19 polypeptide, or pre-FGF-19 polypeptide is purified by immunoaffinity chromatography using antibodies specific for the FGF-19 polypeptide of interest. In general, an immunoaffinity column is constructed by covalently coupling the anti-FGF-19 polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of FGF-19 polypeptide by preparing a fraction from cells containing FGF-19 polypeptide in a soluble form. This preparation is derived by solubilization of the whole cell or of a subcellular fraction obtained via differential centrifugation by the addition of detergent or by other methods well known in the art. Alternatively, soluble FGF-19 polypeptide containing a signal sequence may be secreted in useful quantity into the medium in which the cells are grown.

A soluble FGF-19 polypeptide-containing preparation is passed over the immunoaffinity column, and the column is washed under conditions that allow the preferential absorbance of FGF-19 polypeptide (e.g., high ionic strength buffers in the presence of detergent). Then, the column is eluted under conditions that disrupt antibody/FGF-19 polypeptide binding (e.g., a low pH buffer such as approximately pH 2-3, or a high concentration of a chaotrope such as urea or thiocyanate ion), and FGF-19 polypeptide is collected.

### Example 9

### Drug Screening

This invention is particularly useful for screening compounds by using FGF-19 polypeptides or binding fragment thereof in any of a variety of drug screening techniques. The FGF-19 polypeptide or fragment employed in such a test may either be free in solution, affixed to a solid support, borne on a cell surface, or located intracellularly. One method of drug screening utilizes eukaryotic or prokaryotic host cells which are stably transformed with recombinant nucleic acids expressing the FGF-19 polypeptide or fragment. Drugs are screened against such transformed cells in competitive binding assays. Such cells, either in viable or fixed form, can be used for standard binding assays. One may measure, for example, the formation of complexes between FGF-19 polypeptide or a fragment and the agent being tested. Alternatively, one can examine the diminution in complex formation between the FGF-19 polypeptide and its target cell or target receptors caused by the agent being tested.

Thus, the present invention provides methods of screening for drugs or any other agents which can affect a FGF-19 polypeptide-associated disease or disorder. These methods comprise contacting such an agent with an FGF-19 polypeptide or fragment thereof and assaying (I) for the presence of a complex between the agent and the FGF-19 polypeptide or fragment, or (ii) for the presence of a complex between the FGF-19 polypeptide or fragment and the cell, by methods well known in the art. In such competitive binding assays, the FGF-19 polypeptide or fragment is typically labeled. After suitable incubation, free FGF-19 polypeptide or fragment is separated from that present in bound form, and the amount of free or uncomplexed label is a measure of the ability of the particular agent to bind to FGF-19 polypeptide or to interfere with the FGF-19 polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a FGF-19 polypeptide, the peptide test compounds are reacted with FGF-19 polypeptide and washed. Bound FGF-19 polypeptide is detected by methods well known in the art. Purified FGF-19 polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding FGF-19 polypeptide specifically compete with a test compound for binding to FGF-19 polypeptide or fragments thereof. In this manner, the antibodies can be used to detect the presence of any peptide which shares one or more antigenic determinants with FGF-19 polypeptide.

### Example 10

### Rational Drug Design

The goal of rational drug design is to produce structural analogs of biologically active polypeptide of interest (i.e., a FGF-19 polypeptide) or of small molecules with which they interact, e.g., agonists, antagonists, or inhibitors. Any of these examples can be used to fashion drugs which are more active or stable forms of the FGF-19 polypeptide or which enhance or interfere with the function of the FGF-19 polypeptide in vivo (c.f., Hodgson, Bio/Technology, 9: 19-21 (1991)).

In one approach, the three-dimensional structure of the FGF-19 polypeptide, or of an FGF-19 polypeptide-inhibitor complex, is determined by x-ray crystallography, by computer modeling or, most typically, by a combination of the two approaches. Both the shape and charges of the FGF-19 polypeptide must be ascertained to elucidate the structure and to determine active site(s) of the molecule. Less often, useful information regarding the structure of the FGF-19 polypeptide may be gained by modeling based on the structure of homologous proteins. In both cases, relevant structural information is used to design analogous FGF-19 polypeptide-like molecules or to identify efficient inhibitors. Useful examples of rational drug design may include molecules which have improved activity or stability as shown by Braxton and Wells, Biochemistry 31:7796-7801 (1992) or which act as inhibitors, agonists, or antagonists of native peptides as shown by Athauda et al., J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the FGF-19 polypeptide may be made available to perform such analytical studies as X-ray crystallography. In addition, knowledge of the FGF-19 polypeptide amino acid sequence provided herein will provide guidance to those employing computer modeling techniques in place of or in addition to x-ray crystallography.

### Example 11

### Investigation of Weight, Leptin Levels, Food Intake, Urine Production, Oxygen Consumption, Triglyceride and Free Fatty Acid Levels, and β-3 Adrenergic Receptor in FGF-19 Transgenic Mice

As described herein, FGF-19 has been newly identified as a member of a growing family of secreted proteins related to fibroblast growth factor. FGF-19 has been characterized herein as interacting specifically with FGF receptor 4, and not with other known FGF receptors, and does not appear to act as a mitogen. To further investigate the functions of this protein, transgenic mice have been generated that express human FGF-19.

In particular, the cDNA encoding human FGF-19 was cloned into a plasmid that contains the promoter for myosin light chain. This promoter is sufficient for muscle specific transcription of the transgene. A splice acceptor and donor was also included 5′ to the FGF-19 cDNA to increase the level of expression and a splice donor and acceptor with a poly A addition signal was included 3′ to the FGF-19 cDNA to increase the level of transcription and to provide a transcription termination site.

The DNA encompassing the MLC promoter, the 5′ splice acceptor and donor, the FGF-19 cDNA and the 3′ splice acceptor and donor and the transcription termination site (the transgene) was released from the bacterial vector sequences using appropriate restriction enzymes and purified following size fractionation on agarose gels. The purified DNA was injected into one pronucleus of fertilized mouse eggs and transgenic mice generated and identified as described (Genetic Modification of Animals; Tim Stewart; In Exploring Genetic Mechanisms pp565-598; 1997 Eds M Singer and P Berg; University Science Books; Sausalito, Calif.). Unless otherwise noted mice were maintained on standard lab chow in a temperature and humidity controlled environment. Standard mouse chow was Purina 5010 (Harlen Teklab, Madison Wis.). The high fat (58% kJ fat) and low fat (10.5% kJ fat) isocaloric diets discussed below were based on the diets described by Surwit and colleagues (Surwit, R. S. et al., Metabolism: Clinical & Experimental 44, 645-651 (1995)) and were purchased from Research Diets (New Brunswick N.J.). A 12 hour (6.00 pm/6.00 am) light cycle was used. The mice were 6 weeks of age for the measurements discussed below for water intake, food consumption, urine output and hematocrit. The leptin, triglycerides and free fatty acid measurements were on the same animals at 8 weeks of age.

Insulin and leptin were assayed by ELISA kits (Crystal Chem, Chicago, Ill.). Glucose was assayed either by LIFESCAN Fast Take glucose meter or the glucose oxidase method. All other hormones and serum metabolites were assayed by Anilytics, Inc (Gaithersburg, Md.). Bomb calorimetry of the food and feces for the metabolizable energy calculations was performed by Anilytics, Inc (Gaithersburg, Md.). Fat content in the muscle and liver were assayed using the extraction procedures of Folch et al., Nucleic Acids Research 15, 3185 (1987) and an enzymatic triglyceride reagent kit (Sigma, St Louis, Mo.).

Glucose tolerance tests were performed by injecting each mouse with 35 mg glucose i.p.—the transgenic mice received more glucose on a per body weight basis than the wild type mice. Insulin suppression tests were performed by injecting each mouse with either 0.3 or 0.5 IU insulin/kg i.v.—the transgenic mice received less insulin in absolute terms than the wild type controls. For both the GTT and the IST whole blood glucose was measured at the indicated times using a LIFESCAN Fast Take glucose meter. For the glucose tracer experiments mice were injected i.p. with 1 uCi/g ³H-2-[1,2-3H]deoxyglucose (30 Ci/mmol, NEN, Boston, Mass), 0.25 uCi/g ¹⁴C-sucrose (0.565 Ci/mmol, Amersham, Arlington Heights, Ill.) and 1.25 mg/g glucose. At the indicated times ³H and ¹⁴C were determined in whole blood.

Core body temperature was monitored telemetrically by i.p. of Physiotel body temperature transmitter devices (Data Sciences International, St. Louis). Activity was monitored via the analysis of the frequency with which the mice broke infrared beams that were placed one per inch (X and Y axes).

Unless otherwise noted, all data are presented as the means plus and minus the standard deviations. Comparisons between transgenic and wild type mice were made using an unpaired student's t test. Body mass sensitive parameters are given as a ratio to the body mass raised to the 0.75 power. Even, P. C., et al., Neuroscience and Behavioural Reviews. 18, 435-447 (1994).

As the results discussed below show, the transgenic mice demonstrate increased food intake and increased metabolic rate as evidenced by their rate of oxygen consumption. Despite the increased food intake, these mice weigh significantly less than their non-transgenic littermates. This decreased body weight appears to be a consequence of decreased adiposity which correlates closely with adipose tissue mass in humans and rodents and which is decreased in the transgenic mice. In further support of this, the transgenic mice have normal linear growth as assessed by nose to rump length measurements. They are normal with respect to body temperature, body (bone length) and hematological values. Co-incident with the increased food intake, the transgenic mice have increased urine output. As the mice do not appear to drink more and are not dehydrated as determined by a normal hematocrit, the increased urine output may be derived from the metabolism of the increased food. As FGF-19 decreases adiposity without altering either of muscle mass or long bone formation, FGF-19 is indicated as an effective therapeutic in the treatment of obesity and related conditions.

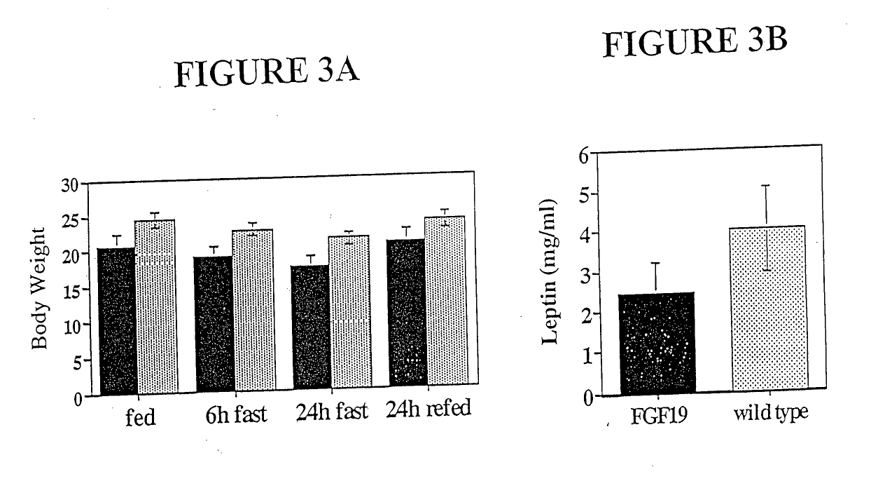

More particularly, MLC-FGF-19 transgenic mice were weighed at various times under different fasting and feeding conditions. More particularly, groups of female FGF-19 transgenic mice and their non-transgenic littermates were weighed at 6 weeks of age during ad libitum feeding, after 6 and 24 hour fasts and 24 hours after ending a 24 hour fast. As shown in FIG. 3A, under all conditions, the FGF-19 transgenic mice (solid bars) weighed less than their wild type, non transgenic littermates (stippled bars).

FIG. 3B shows the sera of the same groups of mice represented in FIG. 3A, assayed for leptin. The decreased leptin in the FGF-19 transgenic mice is consistent with the lower body weights (FIG. 3A) being due to decreased adiposity.

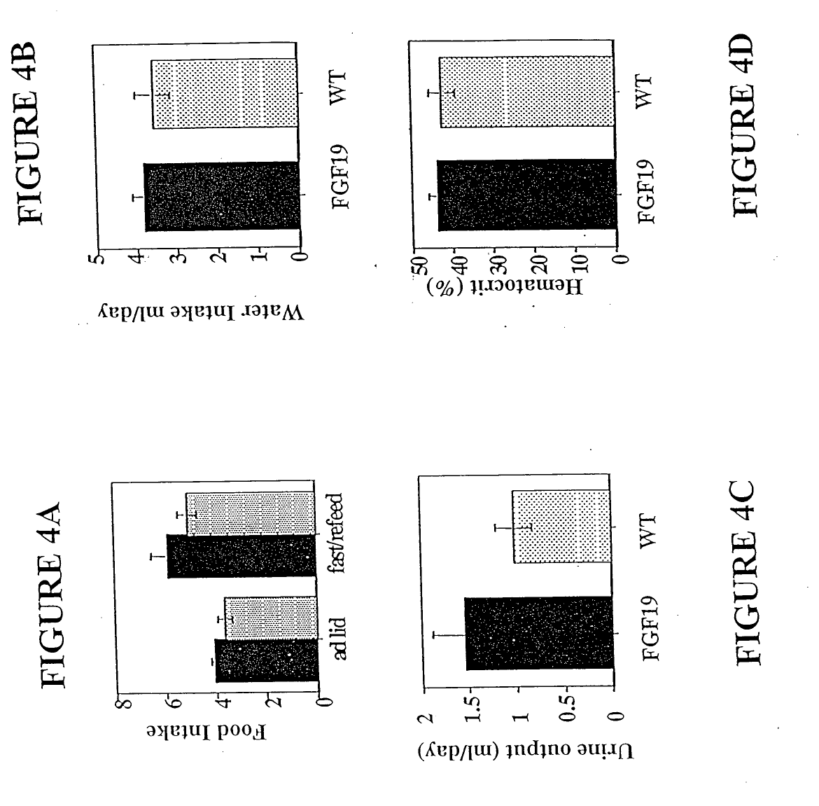

A group of 6 week old transgenic mice were monitored for food intake (FIG. 4A), water intake (FIG. 4B), urine output (FIG. 4C) and hematocrit (FIG. 4D). As can be seen, the FGF-19 transgenic mice (solid bars) consume more food than their wild type littermates but do not drink more. Although there is no change in water consumption, the transgenic mice do produce more urine (FIG. 4C). Despite the increase in urine production, the transgenic mice do not appear to be dehydrated as evidenced by the normal hematocrit (FIG. 4D).

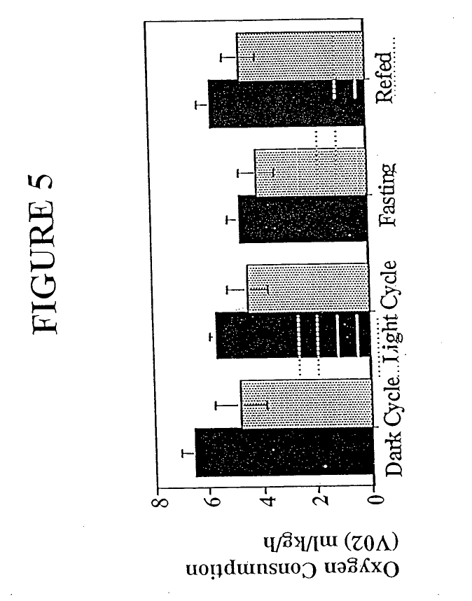

The decrease in body weight (FIG. 3) with an increase in food consumption (FIG. 4) could be explained by an increase in metabolic rate. The metabolic rate was determined by measuring oxygen consumption. As shown in FIG. 5, the FGF-19 transgenic mice have an increased metabolic rate during both light cycles, following a 24 hour fast and 24 hours after ending a 24 hour fast.

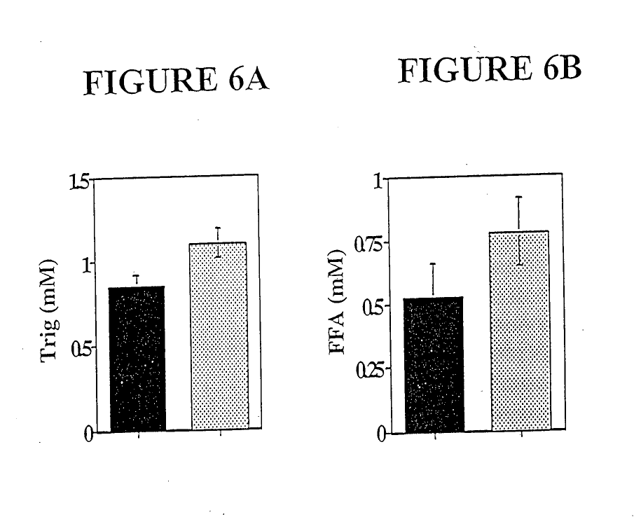

Obesity and elevated triglycerides and free fatty acids are risk factors for cardiovascular disease. As FGF-19 decreases one of the risk factors for cardiovascular disease (obesity (FIG. 3)), it was investigated whether FGF-19 could also lower other risk factors. As can be seen in FIG. 6, the level of triglycerides and free fatty acids (FFA) is also lower in the FGF-19 transgenic mice.

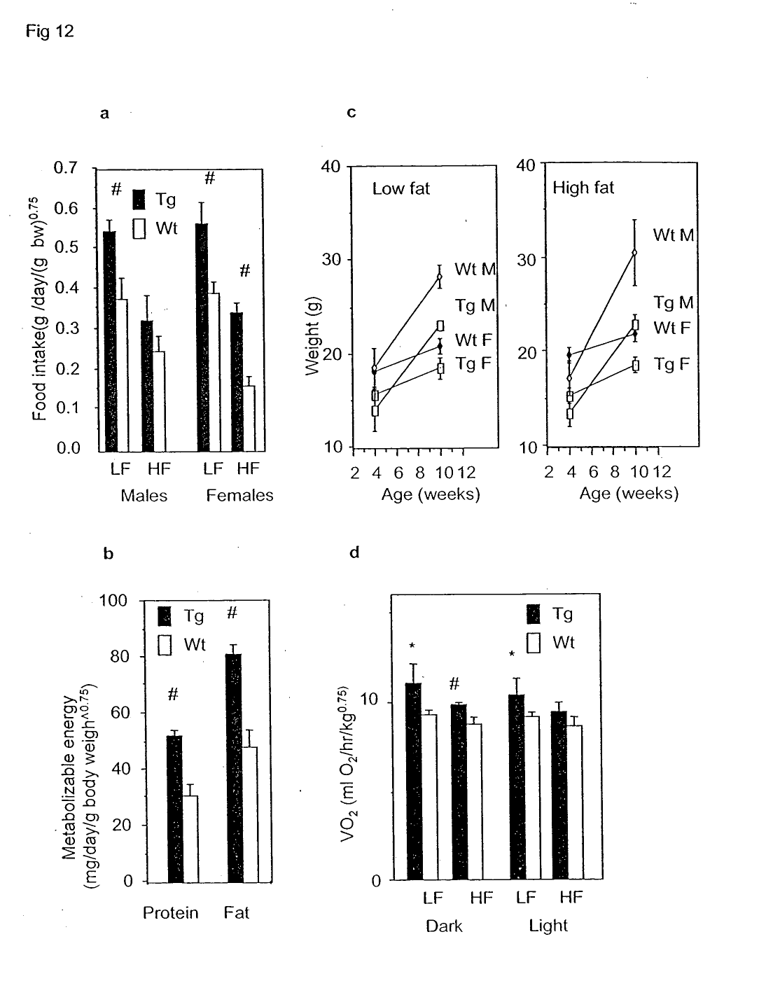

In a separate experiment, on standard lab chow the transgenic mice again weighed less but ate more (see FIG. 12 for mice on defined diets). The decrease in body weight did not appear to be caused by poor nutrient absorption. Calorimetric analysis of the food and feces indicated that the metabolizable energy for the wild type mice on a chow diet was 5811+/−423 kJ/week/kg⁰⁷⁵ vs. 7566+/−557 kJ/week/kg⁰⁷⁵ for the transgenic mice (P<0.001). Rather, the decrease in body weight resulted from an increase in metabolic rate (see FIG. 12 for the metabolic rate of mice on the defined diets). There was no significant difference between the transgenic and the wild type mice with respect to motor activity suggesting that this was not the cause of the increased metabolic rate.

As the mice on a chow diet had a lower body weight and an increased metabolic rate, whether the transgenic mice have an altered response to diet that normally induces obesity was tested. Thus, the mice were fed either an obesity-inducing high fat (HF, 55% fat) diet or one with a lower fat (LF, 11% fat) content. The HF fed wild type mice ate less (in mass) than the LF fed wild type mice, although total caloric intake was not significantly different. The transgenic mice however, ate significantly more than the wild type littermates (FIG. 12a). Metabolizable energy analysis in the mice fed the high fat diet revealed that the transgenic mice also absorbed more fat and protein than the wild type littermates (FIG. 12b). Despite the increase in food intake and metabolizable energy, the transgenic mice (males and females, both HF and LF) weighed less than the wild type littermates (FIG. 12c). As the transgenic mice ate more but weighed less, the metabolic rate in these mice was tested by indirect calorimetry (FIG. 12d). The transgenic mice consumed more oxygen than their wild type littermates on both the high and the low fat diets. The increased oxygen consumption was more marked during the dark cycle, but was also increased relative to the wild type controls during the light cycle.

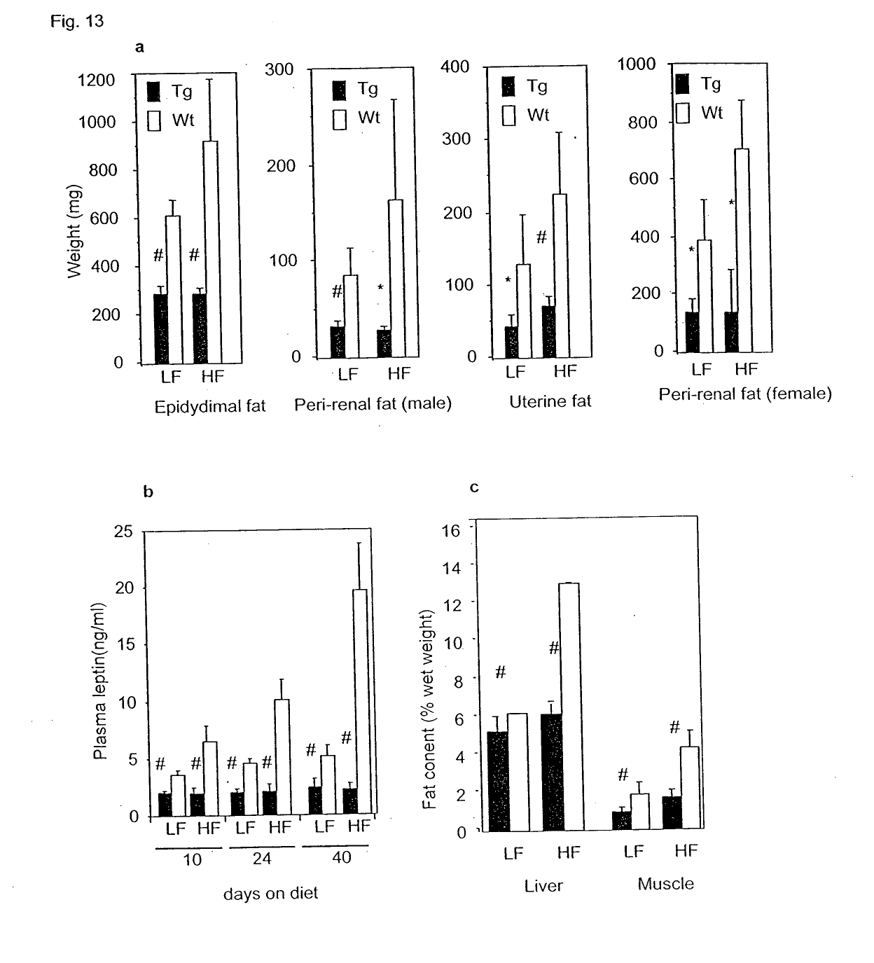

After 6 weeks on the defined diets, the weight and composition of the internal organs were determined. The most striking differences were in the white adipose depots (FIG. 13a). The fat pads from the transgenic mice weighed significantly less than the corresponding fat pads from the wild type littermates. This was the case for both males and females and on both the HF and LF diets. Consistent with the lower adiposity, leptin levels were also lower in the transgenic mice as compared to the wild type mice (FIG. 13b). The high fat diet also led to the expected increase in liver and muscle triglyceride in the control mice (FIG. 13c) whereas the triglyceride content in liver and muscle of the transgenic mice was significantly less (FIG. 13c). The circulating levels of cholesterol and triglycerides were also reduced in the transgenic mice (Table 6).

TABLE 6
Circulating Metabolites and Hormones

| | Triglycerides | Cholesterol | Albumin | Creatinine | ALT |
|---|---|---|---|---|---|
| A. | (g/l) | (mmol/l) | (g/l) | (mol/l) | (U/ml) |
| F Wt | 2.6 +/− 0.2 | 3.2 +/− 0.1 | 37 +/− 5 | 31 +/− 5 | 47 +/− |
| | | | | | 3 |
| F Tr | 2.2 +/− 0.2* | 2.5 +/− 0.2^{#} | 37 +/− 3 | 29 +/− 4 | 52 +/− |
| | | | | | 10 |
| M Wt | 2.4 +/− 0.2 | 4.1 +/− 0.2 | 32 +/− 1 | 28 +/− 4 | 48 +/− |
| | | | | | 3 |
| M Tr | 2.1 +/− 0.2* | 2.6 +/− 0.2^{#} | 35 +/− 1^{#} | 33 +/− 4 | 68 +/− |
| | | | | | 8^{#} |
| | T3 | T4 | Glucagon | Corticosterone | IGF-1 |
| B. | (nmol/l) | (nmol/l) | (ng/l) | (nmol/l) | (g/l) |
| F Wt | 1.2 +/− 0.1 | 40 +/− 4 | 41 +/− 5 | 850 +/− 270 | 720 +/− |
| | | | | | 70 |
| F Tr | 1.2 +/− 0.1 | 40 +/− 1 | 33 +/− 3* | 1070 +/− 360 | 410 +/− |
| | | | | | 50^{#} |
| M Wt | 1.2 +/− 0.1 | 36 +/− 5 | 42 +/− 2 | 510 +/− 90 | 680 +/− |
| | | | | | 60 |
| M Tr | 1.1 +/− 0.1* | 35 +/− 0.3 | 39 +/− 2* | 490 +/− 360 | 420 +/− |
| | | | | | 40^{#} |

All data are from 10 week old mice on regular chow, fasted for four hours; n = 4-5. ^{#}, P < 0.01; *, P < 0.05.

In order to further evaluate the effect of FGF-19 on metabolic rate, various tissues were extracted from FGF-19 transgenic mice. More specifically, brown adipose tissue (BAT), white adipose tissue (WAT) and liver total RNAs extracted from FGF-19 transgenic (TG) mice and wild-type (Wt) mice were used for measuring the relative mRNA abundance of 133-adrenergic receptor. Real-time quantitative RT-PCR (TaqMan analysis) was employed by using primers and probe pairs specific for the 133-adrenergic receptor. The forward primer used was: 5′-CTCCAACATGCCCTATGCG-3′; the reverse primer: ACGAAGAGCATCACAAGGAGG; and the probe: 5′-(FAM) TGCTCTCCTCCTCCGTCTCCTTCTACCTTC (TAMARA)-3′.

Liver, skeletal muscle, WAT and BAT (in rodent and some other species) are the major tissues contributing to whole body metabolic rate. Moreover. the regulation of energy metabolism via the sympathetic nervous system is one of the major regulatory pathways through which the central nervous system (CNS) controls energy metabolism in the peripheral tissues. The neurotransmitters released from the sympathetic nervous system binds to receptors, including the 133-adrenergic receptor, in the peripheral tissues and increase metabolic rate through downstream signaling pathways.

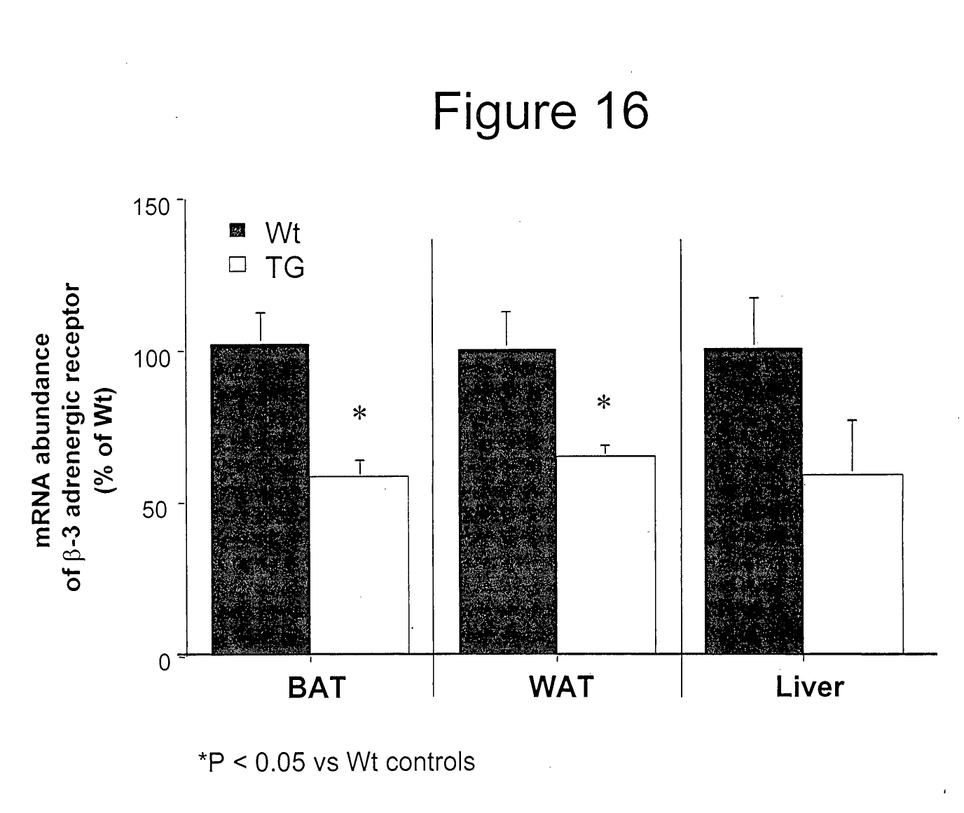

It previously has been documented that expression levels of β3-adrenergic receptor decrease in BAT from mice after exposure to cold environment for 48 hours. Cold exposure increases the activity of the sympathetic nervous system and increases metabolic rate. Therefore, a decreased expression of β3-adrenergic receptor may reflect a negative feedback regulation of the gene expression. The changes in the expression of β3-adrenergic receptor and metabolic rate in FGF-19 transgenic mice are very similar to those in cold-exposed mice. As shown in FIG. 16, a significantly lower expression level of this receptor in BAT and WAT in TG mice vs Wt controls suggests that the sympathetic nervous system is involved in the up-regulation of metabolic rate by FGF 19.

### Example 12

### Recombinant FGF-19 Administration Leads to an Increase in Food Uptake and an Increase in Oxygen Consumption

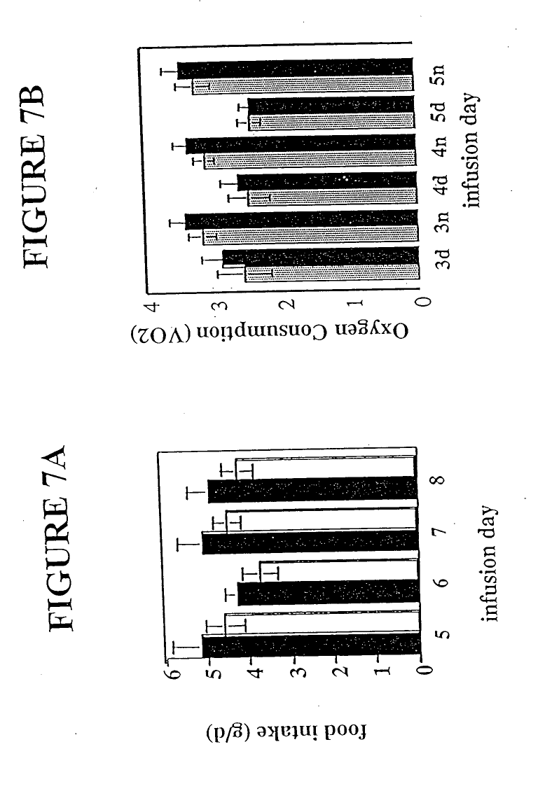

To confirm that the effects seen in the FGF-19 transgenic mice were caused by the FGF-19 protein, groups of non-transgenic FvB mice were infused with recombinant FGF-19 (1 mg/kg/day, iv) delivered by an osmotically driven implanted pump. As shown in FIGS. 7A-B, administration of recombinant human FGF-19 causes an increase in food intake as compared to the mice infused with the carrier alone. In addition, FGF-19 infusion leads to an increase in metabolic rate as measured by oxygen consumption.

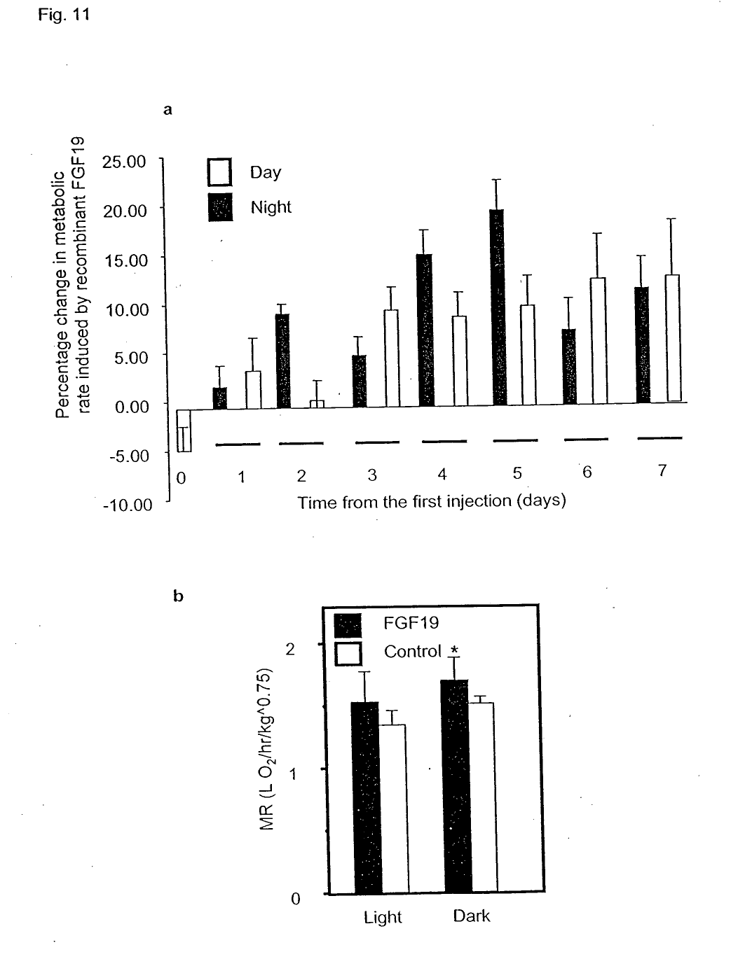

In a separate experiment to confirm the ability of FGF-19 to increase metabolic rate independent of local delivery effects, recombinant human FGF-19, which was expressed in E. Coli (see below), purified, refolded and assayed for its ability to bind to recombinant human FGF receptor-4, was tested in mice. Normal chow fed mice were injected (30 μg/mouse; i.v., twice/day) with recombinant human FGF-19 and their metabolic rate monitored by indirect calorimetry. Oxygen consumption was measured in a Columbus Instruments Oxymax open circuit calorimeter (Columbus, Ohio). The metabolic rate began to increase within 24 of the first injection (FIG. 11A) and, as for the transgenic mice, the effect appeared to be greater during the night. The mean (+/−SD) metabolic rate over days 4, 5 and 6 is shown in FIG. 11B and demonstrates that recombinant FGF-19 delivered systemically is able to significantly increase metabolic rate.

Recombinant HFGF-19 was expressed intracellularly in E. coli by inserting the coding sequence for amino acids 26-216 into a vector downstream of the phoA promoter and upstream of the lambda to transcriptional terminator. (Scholtissek, S. & Grosse, F., Nucleic Acids Research 15, 3185 (1987); Chang, C. N., et al., Gene 55, 189-196 (1987)) Additionally, silent codon changes were designed into the 5′ sequence of hFGF-19 to reduce the likelihood of potential secondary structure formation in the translation initiation region. (Yansura, D. G. & SImmons, Methods. A companion to Methods in Enzymology. 4, 151-158 (1992)). Human FGF-19 was purified via anion exchange chromatography, size exclusion chromatography, and preparative reverse phase chromatography. Sequence analysis and analysis by mass spectrometry indicated that purified recombinant FGF-19 had the expected mass and N-terminal sequence. Binding of the human recombinant FGF-19 to recombinant receptor-4 was measured using I¹²⁵-FGF-19 and IgG tagged FGFr4.

### Example 13

### FGF-19 Decreases Glucose Uptake and Increases Leptin Release from Adipocytes

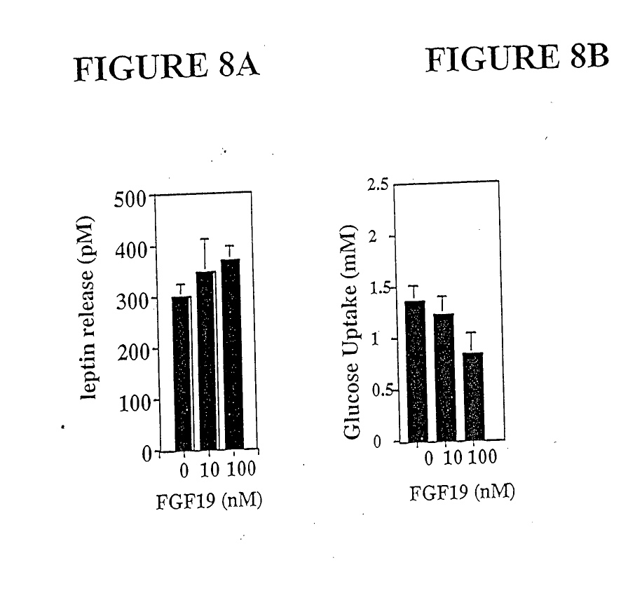

To further investigate the mechanism by which FGF-19 alters metabolism, recombinant human FGF-19 was added to cultures of primary rat adipocytes and glucose uptake and leptin release by the cells was measured. As shown in FIGS. 8A-B, FGF-19 increases the release of leptin from and decreases the uptake of glucose into primary rat adipocytes.

### Example 14

### Investigation of Glucose Tolerance and Fat Pad Weights on FGF-19 Transgenic Mice Fed High Fat Diets

Generally, mice (and humans) on a high fat diet will gain weight and adiposity and will become either glucose intolerant or diabetic. To examine whether exposure to FGF-19 impacts the adiposity and glucose tolerance, cohorts of the transgenic mice and their non transgenic (age and sex matched) littermates were put onto a high fat diet essentially as described by Rebuffe-Scrive et al Metabolism Vol 42, No 11 1993 pp1405-1409 and Surwit et al Metabolism, Vol 44, No 5 1995 pp 645-651 with the modification that the sodium content was normalized with respect to the normal chow (diets prepared by Research Diets Inc. Catalog no. D12330N).

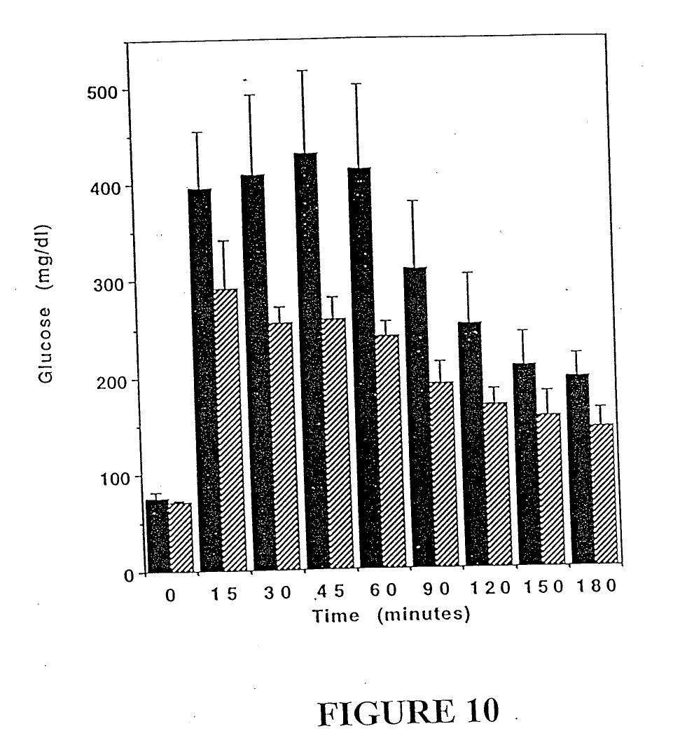

After ten weeks on either the normal mouse chow or on the high fat diet the mice (female transgenic and their non transgenic littermates) were subjected to a glucose tolerance test. Thus each mouse was injected intraperitoneally with 1.0 mg glucose per kg of body weight and the concentration of glucose present in the blood was measured at intervals following the injection. The graph in FIG. 10 shows the glucose levels in the mice and demonstrates that 8/9 of the female non transgenic mice that had been fed the high fat diet would be defined as diabetic (2 hour glucose levels greater than 200 mg/dl; (World Book of Diabetes in Practice. Vo] 3; Ed Krall, L. P.; Elsevier)) whereas 0/5 of the transgenic mice fed a comparable diet would be considered diabetic.

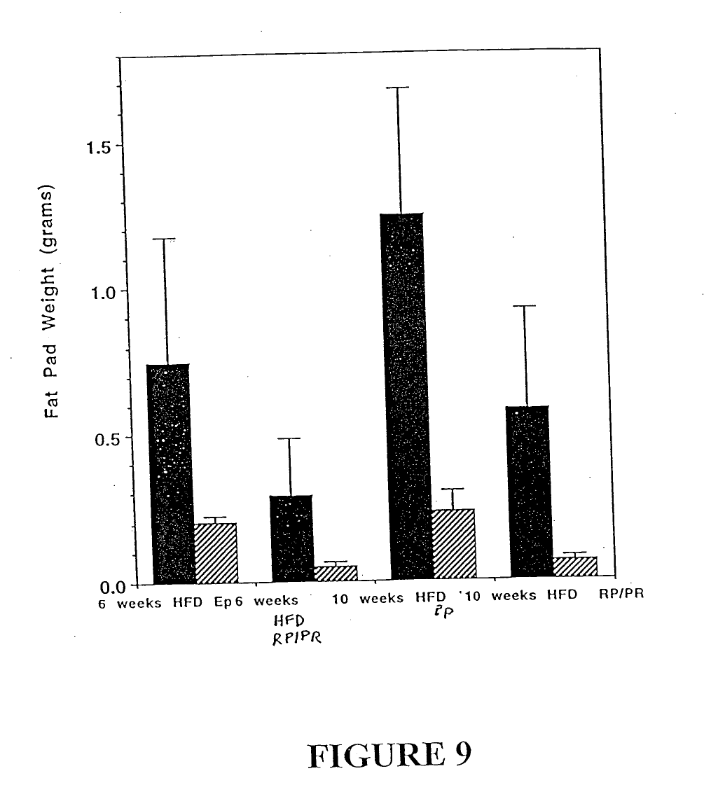

The male mice that were fed the high fat diet were sacrificed after being on the diet for either 6 or 10 weeks and the adiposity determined by measuring the weights of specific fat depots. As is shown in FIG. 9 the transgenic mice that had been fed a high fat diet were significantly less fat then the non transgenic littermates.

### Example 15

### Increased Insulin Sensitivity in Response to FGF-19

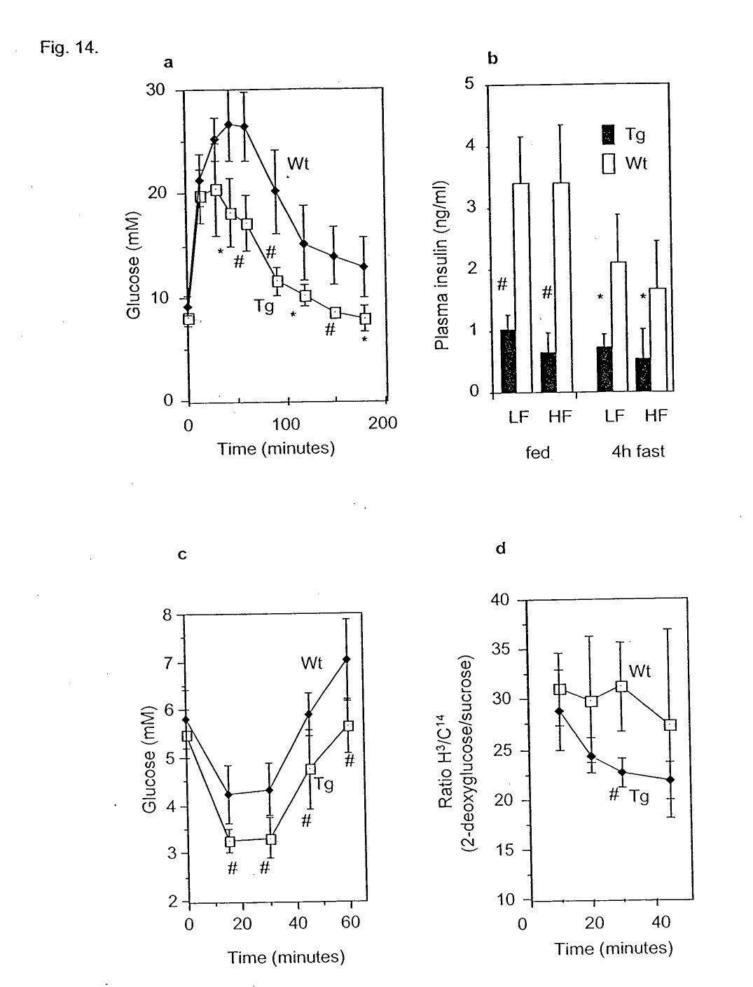

As increased adiposity may contribute to impaired glucose tolerance and susceptibility to type II diabetes, the effect of FGF-19 expression on glucose metabolism was tested. The glucose excursion in the transgenic mice following an intraperitoneal injection of glucose (glucose tolerance test, GTT) was markedly reduced compared to the wild type littermates (FIG. 14a). The GTT profiles were similar in both males and females and in the two dietary conditions. There was also a small (10%) but statistically significant and reproducible decrease in fasting glucose (FIG. 14A). The insulin levels in the fed and fasted transgenic mice were also significantly lower (FIG. 14b). An insulin suppression test (IST) which measures the ability of insulin to lower blood glucose was performed. As there is little effect of the diet on glucose tolerance in either the wild type or transgenic mice, the IST and the tracer disposal experiment (below) were performed on mice fed standard lab chow. An injection of 0.5 IU insulin/kg body weight led to significantly lower glucose levels in the transgenic mice as compared to their wild type siblings (FIG. 14c).

The glucose excursions in the GTT (FIG. 14a) and the IST (FIG. 14c) result from changes in both hepatic glucose output and whole body (primarily muscle) glucose disposal. Glucose disposal can be followed using, as a tracer, a non-metabolizable glucose analogue (2-deoxyglucose). The disappearance of this radioactive tracer from the blood of the transgenic mice was more rapid than that of the wild type littermates (FIG. 14d). These findings—the decreased glucose excursion seen in the GTT, the lower glucose levels attained in the IST, the lower insulin levels and the more rapid disappearance of the glucose tracer—all point to a significant increase in insulin sensitivity in these transgenic mice.

### Example 16

### FGF-19 Acts Directly

FGF-19 does not appear to act by increasing the levels of other major hormones implicated in controlling metabolic rate. Leptin is a circulating hormone that increases fat loss independently of decreased food intake. Leptin is reduced in the FGF-19 transgenic mice (FIG. 13b). Thyroid hormone levels are not increased in the transgenic mice (Table 6), and there is no thyroid hyperplasia. Both growth hormone and IGF-1 have also been implicated in maintaining body composition. Growth hormone levels are not obviously altered while total IGF-1 is reduced (Table 6). Corticosterone is also not altered but there is a reduction in glucagon (Table 6). Thus, the data are more consistent with FGF-19 directly increasing metabolic rate rather than acting via a secondary hormone.

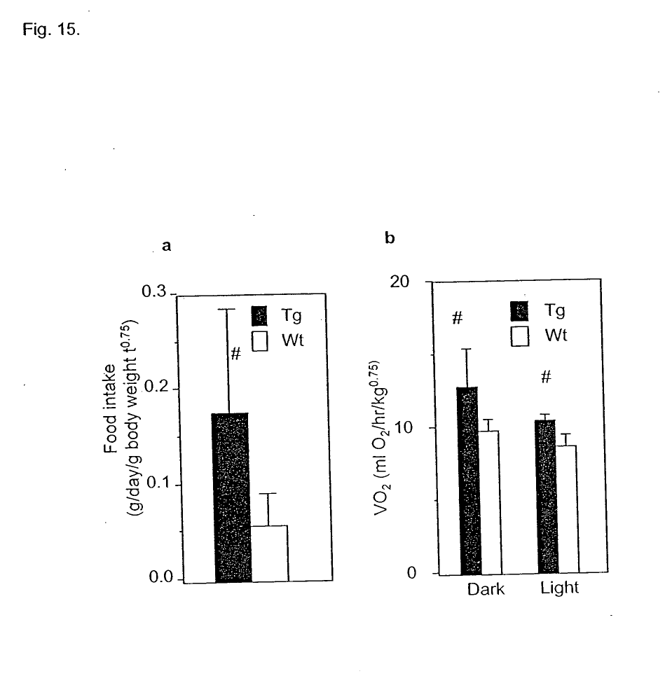

As the myosin light chain (MLC) promoter that was used to drive expression of FGF-19 is used primarily in muscle, a tissue important for metabolic control, it was possible that the site of expression is important for the effects of FGF-19. To address this, transgenic mice that express FGF-19 under the control of the metallothionein promoter were analyzed. Palmiter, R. D. et al., Nature 300, 611-615 (1982). This promoter is used at high levels in the kidney and liver. As shown in FIG. 15, transgenic mice on a high fat diet demonstrated increased food intake (FIG. 15a) and oxygen consumption (FIG. 15b) as well as decreased body and fat pad weights. As the phenotype appears to be independent of promoter, the site of expression does not appear to be critical for the effect of FGF-19. Thus, FGF-19 likely is functioning in an endocrine fashion.

### Example 17

### ICV Administration of FGF-19 Increases Metabolic Rate, Alters Expression Levels of Genes Involved in Energy Metabolism, and Decreases Cyp7a Expression in Liver

Materials and Methods:

Animals. Animals used for the studies were male FVB-NCrBR mice purchased from Charles River, received at 6 weeks of age and housed individually. Mice were cannulated in the lateral ventricle of the brain by the vendor 2 days prior to delivery, and were housed with free access to water and standard rodent chow (Chow 5010, Ralston Purina, St. Louis, Mo.) and with a 12:12 light/dark cycle (lights on 06:00).

Intracerebralventricular (ICV) administration of rhFGF and metabolic rate measurements. Brain-cannulated mice were individually housed in metabolic cages with free access to water and the rodent chow for acclimation for 3 days, and then the baseline of metabolic rate was measured for the following 3 days using indirect calorimetry (oxygen consumption) (Columbus Instruments, Columbus, Ohio). Injection with rhFGF19 at different doses as indicated in figure legends, or 5 μl of vehicle, was carried out at 16:00 each day for 5 days. To facilitate injection, animals were briefly anaesthetized under isoflurane. Animals were then put back to cages for continuous measurement of metabolic rate. On the morning of the sixth day at 10:00, the last injection was performed and the animals were deprived of food. Four to 5 hours later, animals were sacrificed under CO₂, and brain, hypothamalus, liver, and other tissues were harvested and frozen in liquid N₂ and then stored at −70° C. for future gene expression analysis. Since there was no difference on respiratory quotients between controls and rhFGF 19-treated mice, the oxygen consumption rate (VO₂) represents an indicator of the metabolic rate.

The first sample of the measurement after each injection was not included for calculation of the average metabolic rate to rule out any transient effect of the anaesthesia (on the metabolic rate). Daily body weight recorded after each injection was used for calculation of daily average VO₂. Each treated group was composed of at least 4 animals.

Gene expression analysis. Tissue total RNA was extracted by using a total RNA isolation reagent (Biotecx Laboratory, Inc, Houston, Tex.). mRNA abundance was analyzed in total RNA samples treated with DNase as per the manufacture's instructions (Gibco-BRL, Grand Island, N.Y.) and corrected by 18s abundance. Real-time quantitative RT-PCR (TaqMan analysis) was employed by using primers and probe pairs specific for each individual gene to be measured. For NPY, the primers were as follows: forward primer: 5′ CTCCGCTCTGCGACACTACA-3′; reverse primer: 5′-AATCAGTGTCTCAGGGCTGGA-3′; and probe: 5′-(TET) CAATCTCATCACCAGACAGAGATATGGCAAGA(BHQ)-3′. For AgRP, the primers were as follows: forward primer: 5′-GCTAGATCCACAGAACCGCG-3′; reverse primer: 5′-AGCAGGACTCGTGCAGCCT-3′; and probe: 5′-(TET) TCTCGTTCTCCGCGTCGCTGTGT (BHQ)-3′. For POMC, the primers were as follows: forward primer: 5′-GGTGAAGGTGTACCCCAACG-3′; reverse primer: 5′-CCTTCCAGCTCCCTCTTGAA-3′; and probe: 5′-(TET) AGAACGAGTCGGCGGAGGCCTTT (BHQ)-3′. For MC4-R, the primers were as follows: forward primer: 5′-GATTGCTGTCCTCCCAGGC-3′; reverse primer: 5′-TGGTCAAGGTAATCGCCCC-3′; and probe: 5′-(TET) CCATCCGCCAGGGTACCAACATGA(BHQ)-3′. For each treatment, at least 3 independent samples were used for analysis. Each hypothalamus total RNA sample was extracted from hypothalamus pooled from 2 mice.

Results:

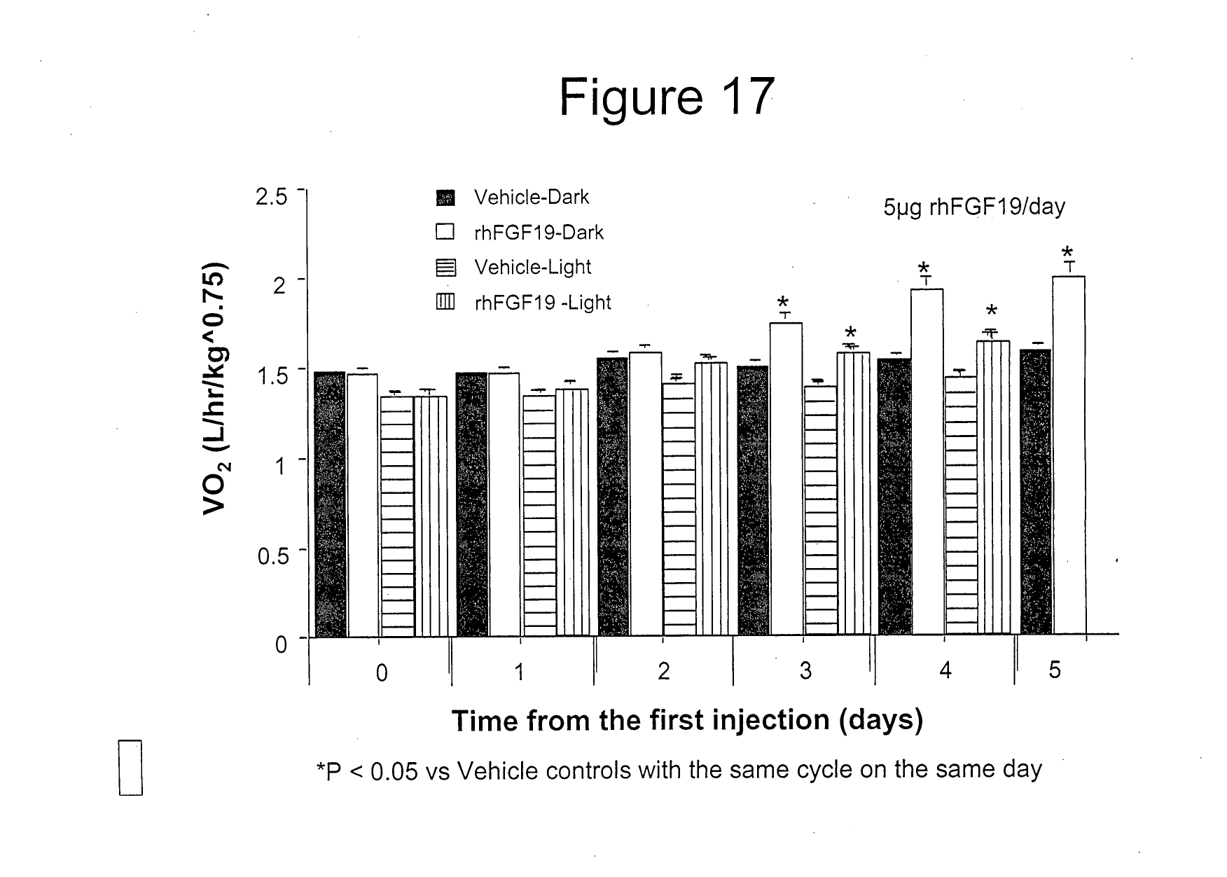

In order to confirm that FGF-19 increases metabolic rate, at least in part, through the CNS, FGF-19 was directly administrated to the CNS for 5 days and the metabolic rate was continuously measured. Male FVB mice with a cannula placed into the lateral ventricle of the brain were given an intracerebralventricular (ICV) dose of vehicle (5 μl) or rhFGF-19 (5 μg), once daily for 5 days. The metabolic rate was measured via indirect calorimetry (oxygen consumption, VO₂). Dark- (1800-0600) and light-cycle (0600-1800) metabolic rate were calculated separately. As shown in FIG. 17, there was no difference of VO₂ between two groups on day 0 (24 hr period prior to 1^{st} injection). From day 3 through day 5, rhFGF-19 administration significantly increased VO₂ vs vehicle, with a relatively larger impact during the dark-cycle (FIG. 17). A greater increase during the dark cycle than the light cycle indicates that the effect of FGF-19 on metabolic rate may be related to feeding behavior (mice eat much more during the dark cycle than the light cycle) or that there might be interactions between FGF-19 and other hormones released during feeding, such as insulin. Because there was no difference on the respiratory quotient (RQ) between the two treatments, a higher VO₂ value indicates that ICV administration of rhFGF 19-19 increases the metabolic rate of the mice. Thus, FGF-19 does increase metabolic rate via the CNS.

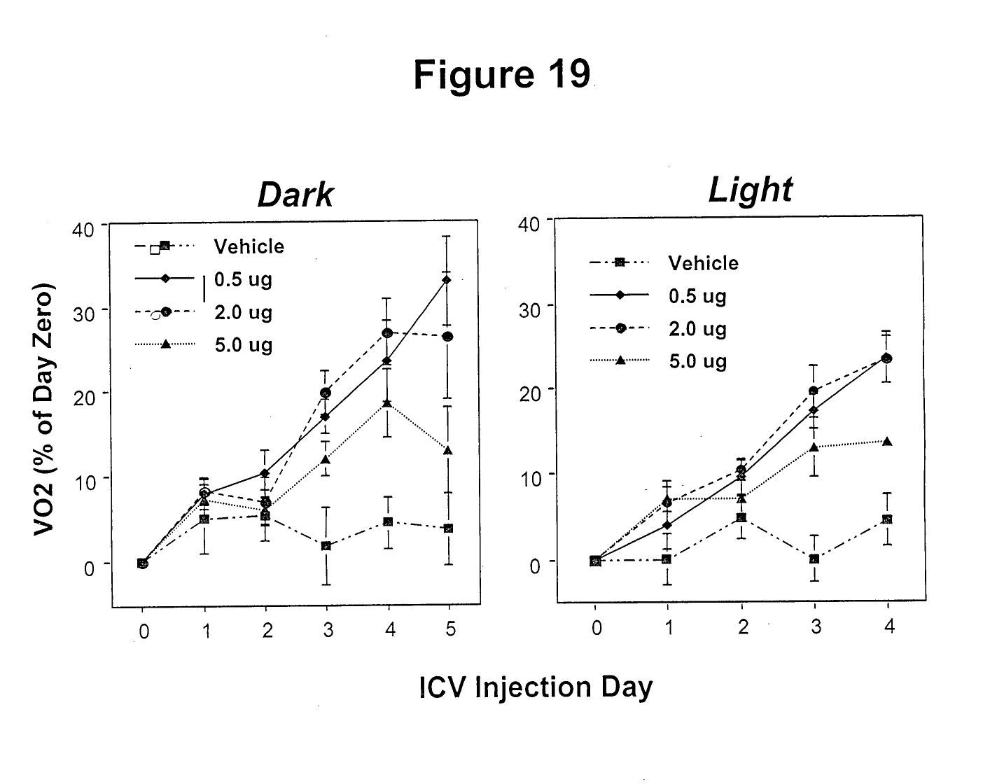

To study the dose response of the metabolic rate to rhFGF-19 and the mechanism of FGF-19 action, different doses of rhFGF-19 were administered to brain. Although three different doses significantly increased the VQvalue, FIG. 19 shows that low doses of rhFGF-19 were especially capable of increasing the metabolic rate of the mice. Specifically, male FVB mice with correct cannula placement into the lateral ventricle were ICV injected with vehicle (5 μl) or different doses of rhFGF 19 (5.0 μg, 2.0 μg, or 0.5 μg), once daily for 5 days. As shown in FIG. 19, from day 3, all rhFGF-19 dosages significantly increased VO₂, with the largest increases at the lowest doses. These data clearly indicate that rhFGF-19 increases metabolic rate, at least in part, through the CNS. Furthermore, since the metabolic rate was unchanged when rhFGF-19 was injected to mice through I.P. or I.V. at the same low doses (2.0 μg/day) for the same period, the increased VO₂ caused by ICV administration of rhFGF-19 is not due to a “leak” of FGF-19 from the brain to the peripheral circulation. These results further demonstrate that FGF-19 increases metabolic rate, at least in part, through the CNS.

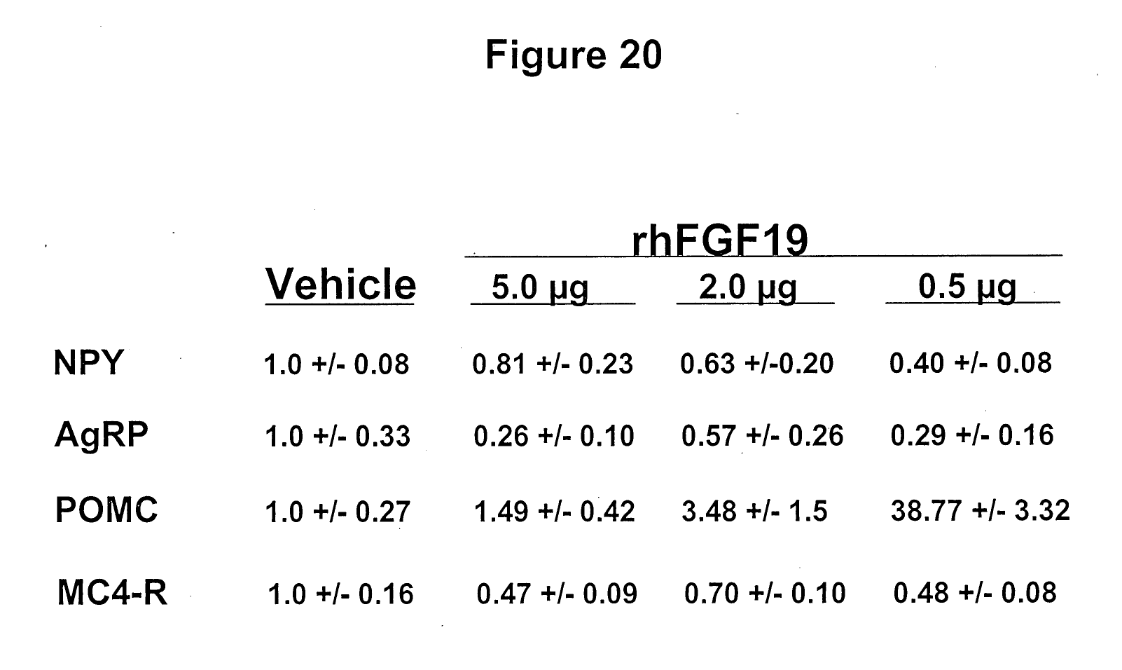

In order to further examine the effects of ICV admininistration of rhFGF-19 on FVB mice, hypothalamustotal RNAs extracted from mice injected with different doses of rhFGF 19 or vehicle (see FIG. 19) were used for measuring the expression of genes associated with energy metabolism via TaqMan Analysis. The hypothalamus is a region of the brain critical for regulation of food intake and thermoregulation. In the arcuate nucleus of hypothalamus, there are two functionally distinct populations of neurons. One population coexpresses NPY and AgRP, and the other coexpresses alpha-MSH (derived from POMC) and CART. It has been well-documented that neuropeptide Y (NPY) and agouti-related protein (AgRP) increase food intake and decrease metabolic rate whereas the product of pro-opiomelanocortin (POMC), melanocortin (MC), decreases food intake and increases metabolic rate. Among other actions, leptin suppresses the expression of NPY and AgRP and induces the expression of alpha-MSH (and CART) through ObRb receptors on arcuate nucleus and thereby decreases the food intake (appetite) and increase metabolic rate (for review, see Spiegelman, B. M. and Flier, J. S. Cell 104, 531-543, 2001). Therefore, decreased expression of the genes encoding NPY and AgRP and increased expression of the gene encoding POMC, as shown in FIG. 20, clearly indicate that rhFGF-19 alters the expression patterns of metabolism-associated genes in the hypothalamus. In addition, decreased expression of MC4-R (FIG. 20) after treatment likely reflects a negative feedback due to an increased MC signaling through the receptor. Therefore, an elevated metabolic rate caused by rhFGF-19 is, among others, through the changes of expression of the genes in hypothalamus which are involved in energy metabolism

Deposit of Material

The following materials have been deposited with the American Type Culture Collection, 10801 University Blvd., Manassas, Va. 20110-2209, USA (ATCC):

| Material | ATCC Dep. No. | Deposit Date |
|---|---|---|
| DNA49435-1219 | 209480 | Nov. 21, 1997 |

This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC § 122 and the Commissioner's rules pursuant thereto (including 37 CFR § 1.14 with particular reference to 8860G 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed is:

1. An isolated nucleic acid molecule which comprises DNA having at least about 80% sequence identity to (a) a DNA molecule encoding an FGF-19 polypeptide comprising the sequence of amino acid residues from about 1 or about 23 to about 216 of FIG. 2 (SEQ ID NO:2), or (b) the complement of the DNA molecule of (a).

2. The isolated nucleic acid molecule of claim 1 comprising the sequence of nucleotide positions from about 464 or about 530 to about 1111 of FIG. 1 (SEQ ID NO:1).

3. The isolated nucleic acid molecule of claim 1 comprising the nucleotide sequence of FIG. 1 (SEQ ID NO:1).

4. The isolated nucleic acid molecule of claim 1 comprising a nucleotide sequence that encodes the sequence of amino acid residues from about 1 or about 23 to about 216 of FIG. 2 (SEQ ID NO:2).

5. An isolated nucleic acid molecule comprising DNA which comprises at least about 80% sequence identity to (a) a DNA molecule encoding the same mature polypeptide encoded by the human protein cDNA deposited with the ATCC on Nov. 21, 1997 under ATCC Deposit No. 209480 (DNA49435-1219), or (b) the complement of the DNA molecule of (a).

6. The isolated nucleic acid molecule of claim 5 comprising DNA encoding the same mature polypeptide encoded by the human protein cDNA deposited with the ATCC on Nov. 21, 1997 under ATCC Deposit No. 209480 (DNA49435-1219).

7. An isolated nucleic acid molecule comprising DNA which comprises at least about 80% sequence identity to (a) the full-length polypeptide coding sequence of the human protein cDNA deposited with the ATCC on Nov. 21, 1997 under ATCC Deposit No. 209480 (DNA49435-1219), or (b) the complement of the coding sequence of (a).

8. The isolated nucleic acid molecule of claim 7 comprising the full-length polypeptide coding sequence of the human protein cDNA deposited with the ATCC on Nov. 21, 1997 under ATCC Deposit No. 209480 (DNA49435-1219).

9. An isolated nucleic acid molecule encoding an FGF-19 polypeptide comprising DNA that hybridizes to the complement of the nucleic acid sequence that encodes amino acids 1 or about 23 to about 216 of FIG. 2 (SEQ ID NO:2).

10. The isolated nucleic acid molecule of claim 9, wherein the nucleic acid that encodes amino acids 1 or about 23 to about 216 of FIG. 2 (SEQ ID NO:2) comprises nucleotides 464 or about 530 to about 1111 of FIG. 1 (SEQ ID NO:1).

11. The isolated nucleic acid molecule of claim 9, wherein the hybridization occurs under stringent hybridization and wash conditions.

12. An isolated nucleic acid molecule comprising at least about 22 nucleotides and which is produced by hybridizing a test DNA molecule under stringent hybridization conditions with (a) a DNA molecule which encodes an FGF-19 polypeptide comprising a sequence of amino acid residues from 1 or about 23 to about 216 of FIG. 2 (SEQ ID NO:2), or (b) the complement of the DNA molecule of (a), and isolating the test DNA molecule.

13. The isolated nucleic acid molecule of claim 12, which has at least about 80% sequence identity to (a) or (b).

14. A vector comprising the nucleic acid molecule of claim 1.

15. The vector of claim 14, wherein said nucleic acid molecule is operably linked to control sequences recognized by a host cell transformed with the vector.

16. A nucleic acid molecule deposited with the ATCC under accession number 209480 (DNA49435-1219).

17. A host cell comprising the vector of claim 14.

18. The host cell of claim 17, wherein said cell is a CHO cell.

19. The host cell of claim 17, wherein said cell is an E. coli.

20. The host cell of claim 17, wherein said cell is a yeast cell.

21. A process for producing an FGF-19 polypeptide comprising culturing the host cell of claim 17 under conditions suitable for expression of said FGF-19 polypeptide and recovering said FGF-19 polypeptide from the cell culture.

22. An isolated FGF-19 polypeptide comprising an amino acid sequence comprising at least about 80% sequence identity to the sequence of amino acid residues from about 1 or about 23 to about 216 of FIG. 2 (SEQ ID NO:2).

23. The isolated FGF-19 polypeptide of claim 22 comprising amino acid residues from about 1 or about 23 to about 216 of FIG. 2 (SEQ ID NO:2).

24. An isolated FGF-19 polypeptide having at least about 80% sequence identity to the polypeptide encoded by the cDNA insert of the vector deposited with the ATCC on Nov. 21, 1997 as ATCC Deposit No. 209480 (DNA49435-1219).

25. The isolated FGF-19 polypeptide of claim 24 which is encoded by the cDNA insert of the vector deposited with the ATCC on Nov. 21, 1997 as ATCC Deposit No. 209480 (DNA49435-1219).

26. An isolated FGF-19 polypeptide comprising the sequence of amino acid residues from about 1 or about 23 to about 216 of FIG. 2 (SEQ ID NO:2), or a fragment thereof sufficient to provide a binding site for an anti-FGF-19 antibody.

27. An isolated polypeptide produced by (i) hybridizing a test DNA molecule under stringent conditions with (a) a DNA molecule encoding an FGF-19 polypeptide comprising the sequence of amino acid residues from 1 or about 23 to about 216 of FIG. 2 (SEQ ID NO:2), or (b) the complement of the DNA molecule of (a), (ii) culturing a host cell comprising said test DNA molecule under conditions suitable for the expression of said polypeptide, and (iii) recovering said polypeptide from the cell culture.

28. The isolated polypeptide of claim 27, wherein said test DNA has at least about 80% sequence identity to (a) or (b).

29. A chimeric molecule comprising an FGF-19 polypeptide fused to a heterologous amino acid sequence.

30. The chimeric molecule of claim 29, wherein said heterologous amino acid sequence is an epitope tag sequence.

31. The chimeric molecule of claim 29, wherein said heterologous amino acid sequence is a Fc region of an immunoglobulin.

32. An antibody which specifically binds to an FGF-19 polypeptide.

33. The antibody of claim 32, wherein said antibody is a monoclonal antibody.

34. The antibody of claim 32, wherein said antibody is a humanized antibody.

35. The antibody of claim 32, wherein said antibody is an antibody fragment.

36. An agonist to an FGF-19 polypeptide.

37. An antagonist to an FGF-19 polypeptide.

38. A composition of matter comprising (a) an FGF-19 polypeptide, (b) an agonist to an FGF-19 polypeptide, (c) an antagonist to an FGF-19 polypeptide, or (d) an anti-FGF-19 antibody in admixture with a pharmaceutically acceptable carrier.

39. A method for screening for a bioactive agent capable of binding to FGF-19 comprising:
a) adding a candidate bioactive agent to a sample of FGF-19; and
b) determining the binding of said candidate agent to said FGF-19, wherein binding indicates a bioactive agent capable of binding to FGF-19.

40. A method for screening for a bioactive agent capable of modulating the activity of FGF-19, said method comprising the steps of:
a) adding a candidate bioactive agent to a sample of FGF-19; and
(b) determining an alteration in the biological activity of FGF-19, wherein an alteration indicates a bioactive agent capable of modulating the activity of FGF-19.

41. A method according to claim 40, wherein said biological activity is decreased uptake of glucose in adipocytes.

42. A method according to claim 40, wherein said biological activity is increased leptin release from adipocytes.

43. A method according to claim 40, wherein said biological activity is binding to FGF receptor 4.

44. A method of identifying a receptor for FGF-19, said method comprising combining FGF-19 with a composition comprising cell membrane material wherein said FGF-19 complexes with a receptor on said cell membrane material, and identifying said receptor as an FGF-19 receptor.

45. The method of claim 44 wherein FGF-19 binds to said receptor, and said method further includes a step of crosslinking said FGF-19 and receptor.

46. The method of claim 44, wherein said composition is a cell.

47. The method of claim 44, wherein said composition is a cell membrane extract preparation.

48. A method of inducing leptin release from adipocyte cells, said method comprising administering FGF-19 to said cells in an amount effective to induce leptin release.

49. The method of claim 48, wherein said FGF-19 is administered as a protein.

50. The method of claim 48, wherein said FGF-19 is administered as a nucleic acid.

51. A method of inducing a decrease in glucose uptake in adipocyte cells, said method comprising administering FGF-19 to said cells in an amount effective to induce a decrease in glucose uptake.

52. The method of claim 51, wherein said FGF-19 is administered as a protein.

53. The method of claim 51, wherein said FGF-19 is administered as a nucleic acid.

54. A method of inducing an increase in insulin sensitivity in cells, said method comprising administering FGF-19 to said cells in an amount effective to induce an increase in insulin sensitivity.

55. The method of claim 54, wherein said FGF-19 is administered as a protein.

56. The method of claim 54, wherein said FGF-19 is administered as a nucleic acid.

57. A method of treating an individual for obesity, said method comprising administering to said individual a composition comprising FGF-19 in an amount effective to treat said obesity.

58. The method of claim 57, wherein said treatment of obesity further results in the treatment of a condition related to obesity.

59. The method of claim 58, wherein said condition is Type II diabetes.

60. The method of claim 57, wherein said FGF-19 is administered as a protein.

61. The method of claim 57, wherein said FGF-19 is administered as a nucleic acid.

62. The method of claim 57, wherein said composition further comprises a pharmaceutical acceptable carrier.

63. The method according to claim 57, wherein said FGF-19 has at least about 85% amino acid sequence identity to the amino acid sequence shown in FIG. 2 (SEQ ID NO:2).

64. A method of reducing total body mass in an individual, said method comprising administering to said individual an effective amount of FGF-19.

65. The method of claim 64, wherein said FGF-19 is administered as a protein.

66. The method of claim 64, wherein said FGF-19 is administered as a nucleic acid.

67. The method of claim 64, wherein said FGF-19 is administered with a pharmaceutical acceptable carrier.

68. The method of claim 64, wherein said reduction in total body mass includes a reduction in fat of said individual.

69. The method according to claim 64, wherein said FGF-19 has at least about 85% amino acid sequence identity to the amino acid sequence shown in FIG. 2 (SEQ ID NO:2).

70. A method of reducing the level of at least one of triglycerides and free fatty acids in an individual, said method comprising administering to said individual an effective amount of FGF-19.

71. The method of claim 70, wherein said FGF-19 is administered as a protein.

72. The method of claim 70, wherein said FGF-19 is administered as a nucleic acid.

73. The method of claim 70, wherein said FGF-19 is administered with a pharmaceutical acceptable carrier.

74. The method according to claim 70, wherein said FGF-19 has at least about 85% amino acid sequence identity to the amino acid sequence shown in FIG. 2 (SEQ ID NO:2).

75. A method of increasing the metabolic rate in an individual, said method comprising administering to said individual an effective amount of FGF-19.

76. The method of claim 75, wherein said FGF-19 is administered as a protein.

77. The method of claim 75, wherein said FGF-19 is administered as a nucleic acid.

78. The method of claim 75, wherein said FGF-19 is administered with a pharmaceutical acceptable carrier.

79. The method according to claim 75, wherein said FGF-19 has at least about 85% amino acid sequence identity to the amino acid sequence shown in FIG. 2 (SEQ ID NO:2).

80. A rodent comprising a genome comprising a transgene encoding FGF-19.

81. A method of modulating the level of neuropeptide Y in a mammal, said method comprising administering to said mammal an effective amount of FGF-19, or an agonist or antagonist thereof.

82. The method of claim 81, wherein said FGF-19 is administered as a protein.

83. The method of claim 81, wherein said FGF-19 is administered as a nucleic acid.

84. The method of claim 81, wherein said FGF-19 is administered with a pharmaceutical acceptable carrier.

85. The method according to claim 81, wherein said FGF-19 has at least about 85% amino acid sequence identity to the amino acid sequence shown in FIG. 2 (SEQ ID NO:2).

86. A method of modulating the level of agouti-related protein in a mammal, said method comprising administering to said mammal an effective amount of FGF-19, or an agonist or antagonist thereof.

87. The method of claim 86, wherein said FGF-19 is administered as a protein.

88. The method of claim 86, wherein said FGF-19 is administered as a nucleic acid.

89. The method of claim 86, wherein said FGF-19 is administered with a pharmaceutical acceptable carrier.

90. The method according to claim 86, wherein said FGF-19 has at least about 85% amino acid sequence identity to the amino acid sequence shown in FIG. 2 (SEQ ID NO:2).

91. A method of modulating the level of pro-opiomelanocortin in a mammal, said method comprising administering to said mammal an effective amount of FGF-19, or an agonist or antagonist thereof.

92. The method of claim 91, wherein said FGF-19 is administered as a protein.

93. The method of claim 91, wherein said FGF-19 is administered as a nucleic acid.

94. The method of claim 91, wherein said FGF-19 is administered with a pharmaceutical acceptable carrier.

95. The method according to claim 91, wherein said FGF-19 has at least about 85% amino acid sequence identity to the amino acid sequence shown in FIG. 2 (SEQ ID NO:2).


SEQUENCE LISTING (20 sequences)

SEQ ID NO: 1 - DNA, 2137 bp, Homo sapiens
gctcccagccaagaacctcggggccgctgcgcggtggggaggagttccccgaaacccggccgctaagcgaggcctcctcctcccgcagatccgaacggcctgggcggggtcaccccggctgggacaagaagccgccgcctgcctgcccgggcccggggagggggctggggctggggccggaggcggggtgtgagtgggtgtgtgcggggggcggaggcttgatgcaatcccgataagaaatgctcgggtgtcttgggcacctacccgtggggcccgtaaggcgctactatataaggctgccggcccggagccgccgcgccgtcagagcaggagcgctgcgtccaggatctagggccacgaccatcccaacccggcactcacagccccgcagcgcatcccggtcgccgcccagcctcccgcacccccatcgccggagctgcgccgagagccccagggaggtgccatgcggagcgggtgtgtggtggtccacgtatggatcctggccggcctctggctggccgtggccgggcgccccctcgccttctcggacgcggggccccacgtgcactacggctggggcgaccccatccgcctgcggcacctgtacacctccggcccccacgggctctccagctgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaagtaactgagaccatgcccgggcctcttcactgctgccaggggctgtggtacctgcagcgtgggggacgtgcttctacaagaacagtcctgagtccacgttctgtttagctttaggaagaaacatctagaagttgtacatattcagagttttccattggcagtgccagtttctagccaatagacttgtctgatcataacattgtaagcctgtagcttgcccagctgctgcctgggcccccattctgctccctcgaggttgctggacaagctgctgcactgtctcagttctgcttgaatacctccatcgatggggaactcacttcctttggaaaaattcttatgtcaagctgaaattctctaattttttctcatcacttccccaggagcagccagaagacaggcagtagttttaatttcaggaacaggtgatccactctgtaaaacagcaggtaaatttcactcaaccccatgtgggaattgatctatatctctacttccagggaccatttgcccttcccaaatccctccaggccagaactgactggagcaggcatggcccaccaggcttcaggagtaggggaagcctggagccccactccagccctgggacaacttgagaattccccctgaggccagttctgtcatggatgctgtcctgagaataacttgctgtcccggtgtcacctgcttccatctcccagcccaccagccctctgcccacctcacatgcctccccatggattggggcctcccaggccccccaccttatgtcaacctgcacttcttgttcaaaaatcaggaaaagaaaagatttgaagaccccaagtcttgtcaataacttgctgtgtggaagcagcgggggaagacctagaaccctttccccagcacttggttttccaacatgatatttatgagtaatttattttgatatgtacatctcttattttcttacattatttatgcccccaaattatatttatgtatgtaagtgaggtttgttttgtatattaaaatggagtttgtttgt

SEQ ID NO: 2 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 3 - DNA, 26 bp, Artificial Sequence
  Primer_bind
atccgcccagatggctacaatgtgta

SEQ ID NO: 4 - DNA, 22 bp, Artificial Sequence
  Primer_bind
ccagtccggtgacaagcccaaa

SEQ ID NO: 5 - DNA, 42 bp, Artificial Sequence
  Synthetic oligonucleotide probe
gcctcccggtctccctgagcagtgccaaacagcggcagtgta

SEQ ID NO: 6 - DNA, 19 bp, Artificial Sequence
  Primer_bind
ctccaacatgccctatgcg

SEQ ID NO: 7 - DNA, 21 bp, Artificial Sequence
  Primer_bind
acgaagagcatcacaaggagg

SEQ ID NO: 8 - DNA, 30 bp, Artificial Sequence
  Synthetic oligonucleotide probe
tgctctcctcctccgtctccttctaccttc

SEQ ID NO: 9 - DNA, 20 bp, Artificial Sequence
  Primer_bind
ctccgctctgcgacactaca

SEQ ID NO: 10 - DNA, 21 bp, Artificial Sequence
  Primer_bind
aatcagtgtctcagggctgga

SEQ ID NO: 11 - DNA, 32 bp, Artificial Sequence
  Synthetic oligonucleotide probe
caatctcatcaccagacagagatatggcaaga

SEQ ID NO: 12 - DNA, 20 bp, Artificial Sequence
  Primer_bind
gctagatccacagaaccgcg

SEQ ID NO: 13 - DNA, 19 bp, Artificial Sequence
  Primer_bind
agcaggactcgtgcagcct

SEQ ID NO: 14 - DNA, 23 bp, Artificial Sequence
  Synthetic Oligonucleotide probe
tctcgttctccgcgtcgctgtgt

SEQ ID NO: 15 - DNA, 20 bp, Artificial Sequence
  Primer_bind
ggtgaaggtgtaccccaacg

SEQ ID NO: 16 - DNA, 20 bp, Artificial Sequence
  Primer_bind
ccttccagctccctcttgaa

SEQ ID NO: 17 - DNA, 23 bp, Artificial Sequence
  Synthetic oligonucleotide probe
agaacgagtcggcggaggccttt

SEQ ID NO: 18 - DNA, 19 bp, Artificial Sequence
  Primer_bind
gattgctgtcctcccaggc

SEQ ID NO: 19 - DNA, 19 bp, Artificial Sequence
  Primer_bind
tggtcaaggtaatcgcccc

SEQ ID NO: 20 - DNA, 24 bp, Artificial Sequence
  Synthetic oligonucleotide probe
ccatccgccagggtaccaacatga